(19) United States
(12) Patent Application Publication
(10) Pub. No.: US 2010/0111853 A1
(43) Pub. Date: May 6, 2010

(54) ANTIBODIES THAT BIND TO IL-12 AND METHODS OF PURIFYING THE SAME

(75) Inventors: Robert K. Hickman, Worcester, MA (US); Qing Huang, Worcester, MA (US); Barbara Perilli-Palmer, Bolton, MA (US)

Correspondence Address: BAKER BOTTS L.L.P., 30 ROCKEFELLER PLAZA, 44th Floor, NEW YORK, NY, 10112-4498, US

(73) Assignee: Abbott Laboratories, Inc., Abbott Park, IL (US)

(21) Appl. No.: 12/582,434

(22) Filed: Oct. 20, 2009

Related U.S. Application Data
(60) Provisional application No. 61/196,752, filed on Oct. 20, 2008.

(51) Int. Cl. A61K 51/10; C07K 1/18; C07K 1/34; A61K 39/395; A61K 38/20; A61P 37/00; A61P 19/02; A61P 29/00; A61P 1/00; A61P 3/10; A61P 11/00; A61P 15/00; A61P 25/00; A61P 9/00

(52) U.S. Cl. 424/1.49; 530/416; 530/414; 424/130.1; 424/158.1; 424/85.2

(57) ABSTRACT

Anti-IL-12 antibodies are disclosed herein, including antigen-binding portions thereof. One or more methods for isolating and purifying anti-IL-12 antibodies from a sample matrix is presented. These isolated anti-IL-12 antibodies can be used in a clinical setting as well as in research and development. Pharmaceutical compositions comprising isolated anti-IL-12 antibodies are also described.

### CROSS REFERENCE TO RELATED APPLICATIONS

This application claims the benefit of U.S. Provisional Application Ser. No. 61/196,752 filed Oct. 20, 2008, which is hereby incorporated by reference in its entirety.

### BACKGROUND OF THE INVENTION

Human interleukin 12 (IL-12) has been characterized as a cytokine with a unique structure and pleiotropic effects. IL-12 plays a critical role in the pathology associated with several diseases involving immune and inflammatory responses. A review of IL-12, its biological activities, and its role in disease can be found in Gately et al. (1998) Ann. Rev. Immunol. 16:495-521.

Structurally, IL-12 is a heterodimeric protein comprising a 35 kDa subunit (p35) and a 40 kDa subunit (p40), which are both linked together by a disulfide bridge (referred to as the “p70 subunit”). The heterodimeric protein is produced primarily by antigen-presenting cells such as monocytes, macrophages, and dendritic cells. These cell types also secrete an excess of the p40 subunit relative to the p70 subunit. The p40 and p35 subunits are genetically unrelated and neither has been reported to possess biological activity, although the p40 homodimer may function as an IL-12 antagonist.

Functionally, IL-12 plays a central role in regulating the balance between antigen specific T helper type (Th1) and type 2 (Th2) lymphocytes. The Th1 and Th2 cells govern the initiation and progression of autoimmune disorders and IL-12 is critical in the regulation of Th1-lymphocyte differentiation and maturation. Cytokines released by the Th1 cells are inflammatory and include interferon-γ (IFNγ), IL-2 and lymphotoxin (LT). Th2 cells secrete IL-4, IL-5, IL-6, IL-10 and IL-13 to facilitate humoral immunity, allergic reactions, and immunosuppression.

Consistent with the preponderance of Th1 responses in autoimmune diseases and the proinflammatory activities of IFNγ, IL-12 may play a major role in the pathology associated with many autoimmune and inflammatory diseases such as rheumatoid arthritis (RA), multiple sclerosis (MS), and Crohn's disease.

Human patients with MS have demonstrated an increase in IL-12 expression as documented by p40 mRNA levels in acute MS plaques. In addition, ex vivo stimulation of antigen-presenting cells with CD40L-expressing T cells from MS patients resulted in increased IL-12 production compared with control T cells, consistent with the observation that CD40/CD40L interactions are potent inducers of IL-12.

Elevated levels of IL-12 p70 have been detected in the synovia of RA patients compared with healthy controls. Cytokine messenger ribonucleic acid (mRNA) expression profile in the RA synovia identified predominantly Th1 cytokines. IL-12 also appears to play a critical role in the pathology associated with Crohn's disease (CD). Increased expression of IFNγ and IL-12 has been observed in the intestinal mucosa of patients with this disease. The cytokine secretion profile of T cells from the lamina propria of CD patients is characteristic of a predominantly Th1 response, including greatly elevated IFNγ levels. Moreover, colon tissue sections from CD patients show an abundance of IL-12 expressing macrophages and IFNγ expressing T cells.

Due to the role of human IL-12 in a variety of human disorders, therapeutic strategies have been designed to inhibit or counteract IL-12 activity. In particular, antibodies that bind to, and neutralize, IL-12 have been sought as a means to inhibit IL-12 activity. It is important that a therapeutic regime comprising antibodies against IL-12 be of high purity. The present invention addresses this need without the use of a Protein A column or an equivalent Protein A-based purification step.

### SUMMARY OF THE INVENTION

In certain embodiments, the present invention is directed to purified, isolated antibodies and antibody fragments that bind to IL-12 as well as pharmaceutical compositions comprising such antibodies and fragments. In certain embodiments, the invention pertains to isolated antibodies, or antigen-binding portions thereof, that bind to human IL-12. The isolated anti-IL-12 antibodies of the present invention can be used in a clinical setting as well as in research and development. In certain embodiments, the present invention is directed to the anti-IL-12 antibody comprising the heavy and light chain sequences identified in SEQ ID NOs. 1 and 2.

Certain embodiments of the invention are directed toward methods of purifying anti-IL-12 antibodies, or antigen-binding portions thereof, from a sample matrix to render them substantially free of host cell proteins (“HCPs”). In certain aspects, the sample matrix (or simply “sample”) comprises a cell line employed to produce anti-IL-12 antibodies of the present invention. In particular aspects, the sample comprises a cell line used to produce human anti-IL-12 antibodies.

In certain embodiments of the present invention a sample matrix comprising the putative anti-IL-12 antibody, or antigen-binding portion thereof, is subjected to a pH adjustment. In certain aspects, the pH is adjusted to about 3.5. The low pH, among other things, promotes the reduction and/or inactivation of pH-sensitive viruses that may be contaminating the sample. After a suitable period of time, the pH is adjusted to approximately 5.0 and the sample is subjected to ion exchange chromatography to produce an eluate. In certain aspects, the ion exchange eluate is collected and further subjected to hydrophobic interactive chromatography to produce an eluate. The hydrophobic interactive chromatography eluate can then be collected for further processing or use.

In certain embodiments the present invention provides for a method of purifying IL-12 antibodies that comprises a primary recovery step to, among other things, remove cells and cellular debris. In certain embodiments of the above-described method, the primary recovery step includes one or more centrifugation or depth filtration steps. For example, and not by way of limitation, such centrifugation steps can be performed at approximately 7000×g to approximately 11,000×g. In addition, certain embodiments of the above-described method will include a depth filtration step, such as a delipid depth filtration step.

In certain embodiments of the above-described method, the ion exchange step can be either cation or anion exchange chromatography, or a combination of both. This step can include multiple ion exchange steps such as a cation exchange step followed by an anion exchange step or visa versa. In certain aspects, the ion exchange step involves a two step ion exchange process. Such two step processes can be accomplished, for example, and not by way of limitation, by a first cation exchange step, followed by a second anion exchange step. An exemplary cation exchange column is a column whose stationary phase comprises anionic groups, such as a CM Hyper DF™ column. This ion exchange capture chromatography step facilitates the isolation of the anti-IL-12 antibodies from the primary recovery mixture. A suitable anion exchange column is a column whose stationary phase comprises cationic groups. An example of such a column is a Q Sepharose™ column. One or more ion exchange step further isolates anti-IL-12 antibodies by reducing impurities as host cell proteins and DNA and, where applicable, affinity matrix protein. This anion exchange procedure is a flow through mode of chromatography wherein the anti-IL-12 antibodies do not interact or bind to the anion exchange resin (or solid phase). However, many impurities do interact with and bind to the anion exchange resin.

In certain embodiments, a first and second ion exchange step is performed following primary recovery. In certain of such embodiments, the ion exchange sample is subjected to an intermediate filtration step, either prior to the first ion exchange step, between the two ion exchange steps, or both. In certain aspects, this filtration step comprises capture ultrafiltration/diafiltration (“UF/DF”). Among other activities, such filtration facilitates the concentration and buffer exchange of anti-IL-12 antibodies and antigen-binding portions thereof.

Certain embodiments of the invention provide for a method comprising one or more hydrophobic interactive chromatography (“HIC”) step. A suitable HIC column is one whose stationary phase comprises hydrophobic groups. A non-limiting example of such a column is a Phenyl HP Sepharose™ column. In certain circumstances anti-IL-12 antibodies will form aggregates during the isolation/purification process. Inclusion of one or more HIC step facilitates the reduction or elimination of such aggregations. HIC also assists in the removal of impurities. In certain embodiments the HIC step employs a high salt buffer to promote interaction of the anti-IL-12 antibodies (or aggregations thereof) with the hydrophobic column. The anti-IL-12 antibodies can then be eluted using lower concentrations of salt.

In certain embodiments, the HIC eluate is filtered using a viral removal filter such as, but not limited to, an Ultipor DV50™ filter (Pall Corporation, East Hills, N.Y.). Alternative filters, such as Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, Ill.), can also be used in such embodiments.

In certain embodiments, the invention is directed to one or more pharmaceutical composition comprising an isolated anti-IL-12 antibody or antigen-binding portion thereof and an acceptable carrier. In one aspect, the composition further comprises one or more antibody or antigen-binding portion thereof in addition to the anti-IL-12 antibody. In another aspect, the compositions further comprise one or more pharmaceutical agents.

### BRIEF DESCRIPTION OF THE DRAWINGS

FIG. 1. FIG. 1 discloses the heavy and light chain variable region sequences of a non-limiting example of an anti-IL-12 antibody (ABT-847).

### DETAILED DESCRIPTION OF THE INVENTION

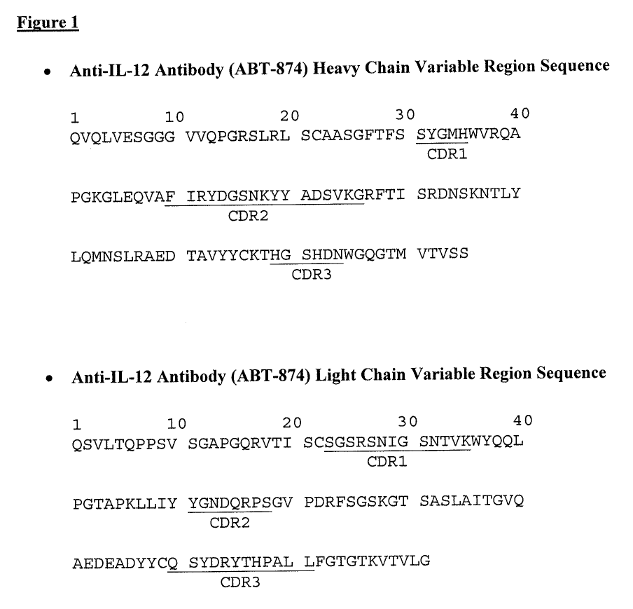

The present invention is directed to antibodies that bind to IL-12. In one aspect, the invention pertains to isolated antibodies, or antigen-binding portions thereof, that bind to human IL-12. The isolated anti-IL-12 antibody of the present invention can be used in a clinical setting as well as in research and development. The present invention also pertains to methods for purifying anti-IL-12 antibodies, or antigen-binding portions thereof. Suitable anti-IL-12 antibodies that may be purified in the context of the instant invention are disclosed in U.S. Pat. No. 6,914,128 (which is hereby incorporated by reference in its entirety) including, but not limited to the anti-IL-12 antibody identified in that patent as J695, and which has subsequently been identified as ABT-874. The sequences of the heavy and light chain variable regions of ABT-874 are set forth in FIG. 1 and SEQ ID NOs. 1 and 2. The present invention also relates to pharmaceutical compositions comprising the anti-IL-12 antibodies or antigen-binding portions thereof described herein.

For clarity and not by way of limitation, this detailed description is divided into the following sub-portions:

1. Definitions;

2. Antibody Generation;

3. Antibody Production;

4. Antibody Purification;

5. Methods of Assaying Sample Purity;

6. Further Modifications;

7. Pharmaceutical Compositions; and

8. Antibody Uses.

### 1. Definitions

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen (e.g., hIL-12). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)₂ fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The phrase “human interleukin 12” (abbreviated herein as hIL-12, or IL-12), as used herein, includes a human cytokine that is secreted primarily by macrophages and dendritic cells. The term includes a heterodimeric protein comprising a 35 kD subunit (p35) and a 40 kD subunit (p40) which are linked together with a disulfide bridge. The heterodimeric protein is referred to as a “p70 subunit”. The structure of human IL-12 is described further in, e.g., Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237, the entire teachings of which are incorporated herein by reference. The nucleic acid encoding IL-12 is available as GenBank Accession No. NM_{—}000882 and the polypeptide sequence is available as GenBank Accession No. NP_{—}000873.2. The term human IL-12 is intended to include recombinant human IL-12 (rh IL-12), which can be prepared by standard recombinant expression methods.

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the gemiline of another mammalian species, such as a mouse, have been grafted onto human framework sequences.

The phrase “selective mutagenesis approach” includes a method of improving the activity of an antibody by selecting and individually mutating CDR amino acids at least one suitable selective mutagenesis position, hypermutation, and/or contact position. A “selectively mutated” human antibody is an antibody which comprises a mutation at a position selected using a selective mutagenesis approach. In another aspect, the selective mutagenesis approach is intended to provide a method of preferentially mutating selected individual amino acid residues in the CDR1, CDR2 or CDR3 of the heavy chain variable region (hereinafter H1, H2, and H3, respectively), or the CDR1, CDR2 or CDR3 of the light chain variable region (hereinafter referred to as L1, L2, and L3, respectively) of an antibody. Amino acid residues may be selected from selective mutagenesis positions, contact positions, or hypermutation positions. Individual amino acids are selected based on their position in the light or heavy chain variable region. It should be understood that a hypermutation position can also be a contact position. In one aspect, the selective mutagenesis approach is a “targeted approach”. The language “targeted approach” is intended to include a method of mutating selected individual amino acid residues in the CDR1, CDR2 or CDR3 of the heavy chain variable region or the CDR1, CDR2 or CDR3 of the light chain variable region of an antibody in a targeted manner, e.g., a “Group-wise targeted approach” or “CDR-wise targeted approach”. In the “Group-wise targeted approach”, individual amino acid residues in particular groups are targeted for selective mutations including groups I (including L3 and H3), II (including H2 and L1) and III (including L2 and H1), the groups being listed in order of preference for targeting. In the “CDR-wise targeted approach”, individual amino acid residues in particular CDRs are targeted for selective mutations with the order of preference for targeting as follows: H3, L3, H2, L1, H1 and L2. The selected amino acid residue is mutated, e.g., to at least two other amino acid residues, and the effect of the mutation on the activity of the antibody is determined. Activity is measured as a change in the binding specificity/affinity of the antibody, and/or neutralization potency of the antibody. It should be understood that the selective mutagenesis approach can be used for the optimization of any antibody derived from any source including phage display, transgenic animals with human IgG germline genes, human antibodies isolated from human B-cells. The selective mutagenesis approach can be used on antibodies which can not be optimized further using phage display technology. It should be understood that antibodies from any source including phage display, transgenic animals with human IgG germline genes, human antibodies isolated from human B-cells can be subject to back-mutation prior to or after the selective mutagenesis approach.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities (e.g., an isolated antibody that specifically binds hIL-12 is substantially free of antibodies that specifically bind antigens other than hIL-12). An isolated antibody that specifically binds hIL-12 may bind IL-12 molecules from other species. Moreover, an isolated antibody may be substantially free of other cellular material and/or chemicals.

A “neutralizing antibody” (or an “antibody that neutralized hIL-12 activity”) includes an antibody whose binding to hIL-12 results in inhibition of the biological activity of hIL-12. This inhibition of the biological activity of hIL-12 can be assessed by measuring one or more indicators of hIL-12 biological activity, such as inhibition of human phytohemagglutinin blast proliferation in a phytohemagglutinin blast proliferation assay (PHA), or inhibition of receptor binding in a human IL-12 receptor binding assay. These indicators of hIL-12 biological activity can be assessed by one or more of several standard in vitro or in vivo assays known in the art.

The term “activity” includes activities such as the binding specificity/affinity of an antibody for an antigen, e.g., an anti-hIL-12 antibody that binds to an IL-12 antigen and/or the neutralizing potency of an antibody, e.g., an anti-hIL-12 antibody whose binding to hIL-12 inhibits the biological activity of hIL-12, e.g., inhibition of PHA blast proliferation or inhibition of receptor binding in a human IL-12 receptor binding assay.

The phrase “surface plasmon resonance” includes an optical phenomenon that allows for the analysis of real-time biospecific interactions by detection of alterations in protein concentrations within a biosensor matrix, e.g., using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., el al. (1995) J. Mol. Recognit. 8:125-131; and Johnnson, B., et al. (1991) Anal. Biochem. 198:268-277, the entire teachings of which are incorporated herein.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The phrase “nucleic acid molecule” includes DNA molecules and RNA molecules. A nucleic acid molecule may be single-stranded or double-stranded, but in one aspect is double-stranded DNA.

The phrase “isolated nucleic acid molecule,” as used herein in reference to nucleic acids encoding antibodies or antibody portions (e.g., VH, VL, CDR3) that bind hIL-12 (including “isolated antibodies”), includes a nucleic acid molecule in which the nucleotide sequences encoding the antibody or antibody portion are free of other nucleotide sequences encoding antibodies or antibody portions that bind antigens other than hIL-12, which other sequences may naturally flank the nucleic acid in human genomic DNA. Thus, e.g, an isolated nucleic acid of the invention encoding a VH region of an anti-IL-12 antibody contains no other sequences encoding other VH regions that bind antigens other than IL-12. The phrase “isolated nucleic acid molecule” is also intended to include sequences encoding bivalent, bispecific antibodies, such as diabodies in which VH and VL regions contain no other sequences other than the sequences of the diabody.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified antibody product is less than the ID50 (the amount of virus that will infect 50 percent of a target population) for that virus, preferably at least 10-fold less than the ID50 for that virus, more preferably at least 100-fold less than the ID50 for that virus, and still more preferably at least 1000-fold less than the ID50 for that virus.

The phrase “contact position” includes an amino acid position in the CDR1, CDR2 or CDR3 of the heavy chain variable region or the light chain variable region of an antibody which is occupied by an amino acid that contacts antigen in one of the twenty-six known antibody-antigen structures. If a CDR amino acid in any of the twenty-six known solved structures of antibody-antigen complexes contacts the antigen, then that amino acid can be considered to occupy a contact position. Contact positions have a higher probability of being occupied by an amino acid which contact antigens than in a non-contact position. In one aspect, a contact position is a CDR position which contains an amino acid that contacts antigen in greater than 3 of the 26 structures (>1.5%). In another aspect, a contact position is a CDR position which contains an amino acid that contacts antigen in greater than 8 of the 25 structures (>32%).

### 2. Antibody Generation

The term “antibody” as used in this section refers to an intact antibody or an antigen binding fragment thereof.

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody preferably can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies of this disclosure are human monoclonal antibodies. Such human monoclonal antibodies directed against IL-12 can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise anti-IL-12 antibodies of this disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-IL-12 antibodies of this disclosure.

Recombinant human antibodies of the invention, including anti-IL-12 antibodies or an antigen binding portion thereof, or anti-IL-12-related antibodies disclosed herein can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In one embodiment, the methods of the invention include anti-IL-12 antibodies and antibody portions, anti-IL-12-related antibodies and antibody portions, and human antibodies and antibody portions with equivalent properties to anti-IL-12 antibodies, such as high affinity binding to hIL-12 with low dissociation kinetics and high neutralizing capacity. In one aspect, the invention provides treatment with an isolated human antibody, or an antigen-binding portion thereof, that dissociates from hIL-12 with a K_{d} of about 1×10⁻⁸ M or less and a K_{off} rate constant of 1×10⁻³ s⁻¹ or less, both determined by surface plasmon resonance. In specific non-limiting embodiments, an anti-IL12 antibody purified according to the invention competitively inhibits binding of ABT-874 to IL12 under physiological conditions.

In yet another embodiment of the invention, anti-IL-12 antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### 3. Antibody Production

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. For example, one approach to converting the anti-IL-12 antibody or anti-IL-12 antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr⁻ host cells with methotrexate selection/amplification) and the neo gene (for G418 selection).

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr- CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to IL-12, specifically hIL-12. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than IL-12 by crosslinking an antibody of the invention to a second antibody by standard chemical crosslinking methods.

In a suitable system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon or Millipore Pellicon ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon or Millipore Pellicon ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 4. Antibody Purification

4.1 Antibody Purification Generally

The invention provides a method for producing a purified (or “HCP-reduced”) antibody preparation from a mixture comprising an antibody and at least one HCP. The purification process of the invention begins at the separation step when the antibody has been produced using methods described above and conventional methods in the art. Typically in the art, antibody-HCP mixtures are subjected to protein A capture (e.g., a protein A column) as an initial purification step, since the antibody binds to protein A whereas HCP will flow through. The purification methods of the present invention have the advantage that it is not necessary to subject the mixture comprising an antibody and at least one HCP to protein A capture (e.g., a protein A column) as an initial step, or as any step in the purification method. Table 1 summarizes one embodiment of a purification scheme. Variations of this scheme are envisaged and are within the scope of this invention.

TABLE 1
Purification steps with their associated purpose

| Purification step | Purpose |
|---|---|
| Primary recovery | clarification of sample matrix |
| Cation exchange | antibody capture, host cell protein and associated |
| chromatography | impurity reduction |
| ultrafiltration/ | concentration and buffer exchange |
| diafiltration | |
| Anion exchange | reduction of host cell proteins and DNA |
| chromatography | |
| Phenyl Sepharose HP | reduction of antibody aggregates and host |
| chromatography | cell proteins |
| Viral filtration | removal of large viruses, if present |
| Final ultrafiltration/ | concentrate and formulate antibody |
| diafiltration | |

Once a clarified solution or mixture comprising the antibody has been obtained, separation of the antibody from the other proteins produced by the cell, such as HCPs, is performed using a combination of different purification techniques, including ion exchange separation step(s) and hydrophobic interaction separation step(s). The separation steps separate mixtures of proteins on the basis of their charge, degree of hydrophobicity, or size. In one aspect of the invention, separation is performed using chromatography, including cationic, anionic, and hydrophobic interaction. Several different chromatography resins are available for each of these techniques, allowing accurate tailoring of the purification scheme to the particular protein involved. The essence of each of the separation methods is that proteins can be caused either to traverse at different rates down a column, achieving a physical separation that increases as they pass further down the column, or to adhere selectively to the separation medium, being then differentially eluted by different solvents. In some cases, the antibody is separated from impurities when the impurities specifically adhere to the column and the antibody does not, i.e., the antibody is present in the flow through.

As noted above, accurate tailoring of a purification scheme relies on consideration of the protein to be purified. In certain embodiments, the separation steps of the instant invention are employed to separate an antibody from one or more HCPs. Antibodies that can be successfully purified using the methods described herein include, but are not limited to, human IgA₁, IgA₂, IgD, IgE, IgG₁, IgG₂, IgG₃, IgG₄, and IgM antibodies. In certain embodiments, the purification strategies of the instant invention exclude the use of Protein A affinity chromatography. Such embodiments are particularly useful for the purification of IgG₃ antibodies, as IgG₃ antibodies are known to bind to Protein A inefficiently. Other factors that allow for specific tailoring of a purification scheme include, but are not limited to: the presence or absence of an Fc region (e.g., in the context of full length antibody as compared to an Fab fragment thereof); the particular germline sequences employed in generating to antibody of interest; and the amino acid composition of the antibody (e.g., the primary sequence of the antibody as well as the overall charge/hydrophobicity of the molecule). Antibodies sharing one or more characteristic can be purified using purification strategies tailored to take advantage of that characteristic.

4.2 Primary Recovery

The initial steps of the purification methods of the present invention involve the first phase of clarification and primary recovery of anti-IL-12 antibody from a sample matrix. In addition, the primary recovery process can also be a point at which to inactivate viruses that can be present in the sample matrix. For example, any one or more of a variety of methods of viral inactivation can be used during the primary recovery phase of purification including heat inactivation (pasteurization), pH inactivation, solvent/detergent treatment, UV and y-ray irradiation and the addition of certain chemical inactivating agents such as β-propiolactone or e.g., copper phenanthroline as in U.S. Pat. No. 4,534,972, the entire teaching of which is incorporated herein by reference. In certain embodiments of the present invention, the sample matrix is exposed to pH viral inactivation during the primary recovery phase.

Methods of pH viral inactivation include, but are not limited to, incubating the mixture for a period of time at low pH, and subsequently neutralizing the pH and removing particulates by filtration. In certain embodiments the mixture will be incubated at a pH of 2 to 5, preferably at a pH of 3 to 4, and more preferably at a pH of 3.5. The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to two 2 hr, preferably 0.5 hr to 1.5 hr, and more preferably the duration will be 1 hr. Virus inactivation is dependent on these same parameters in addition to protein concentration, which may reduce inactivation at high concentrations. Thus, the proper parameters of protein concentration, pH, and duration of inactivation can be selected to achieve the desired level of viral inactivation.

In certain embodiments viral inactivation can be achieved via the use of suitable filters. A non-limiting example of a suitable filter is the Ultipor DV50™ filter from Pall Corporation. Although certain embodiments of the present invention employ such filtration during the primary recovery phase, in other embodiments it is employed at other phases of the purification process, including as either the penultimate or final step of purification. In certain embodiments, alternative filters are employed for viral inactivation, such as, but not limited to, Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, Ill.).

In those embodiments where viral inactivation is employed, the sample mixture can be adjusted, as needed, for further purification steps. For example, following low pH viral inactivation the pH of the sample mixture is typically adjusted to a more neutral pH, e.g., from about 5.0 to about 8.5 prior to continuing the purification process. Additionally, the mixture may be flushed with water for injection (WFI) to obtain a desired conductivity.

In certain embodiments, the primary recovery will include one or more centrifugation steps to further clarify the sample matrix and thereby aid in purifying the anti-IL-12 antibodies. Centrifugation of the sample can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification.

In certain embodiments, the primary recovery will include the use of one or more depth filtration steps to further clarify the sample matrix and thereby aid in purifying the anti-IL-12 antibodies. Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can be a delipid depth filtration step. Although certain embodiments employ depth filtration steps only during the primary recovery phase, other embodiments employ depth filters, including delipid depth filters, during one or more additional phases of purification. Non-limiting examples of depth filters that can be used in the context of the instant invention include the Cuno™ model 30/60ZA depth filters (3M Corp.), and 0.45/0.2 μm Sartopore™ bi-layer filter cartridges.

4.3 Ion Exchange Chromatography

In certain embodiments, the instant invention provides methods for producing a HCP-reduced antibody preparation from a mixture comprising an antibody and at least one HCP by subjecting the mixture to at least one ion exchange separation step such that an eluate comprising the antibody is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

In performing the separation, the initial antibody mixture can be contacted with the ion exchange material by using any of a variety of techniques, e.g., using a batch purification technique or a chromatographic technique.

For example, in the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the HCP(s) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb HCPs that have bound to the ion exchange material. In order to elute bound polypeptides, the salt concentration of the buffer can be increased.

Ion exchange chromatography may also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl(QAE) and quaternary amine(Q) groups. Cationic substitutents include carboxymethyl (CM), sulfoethyl(SE), sulfopropyl(SP), phosphate(P) and sulfonate(S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP- SEPHADEX® and DEAE-, Q-, CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa.

A mixture comprising an antibody and impurities, e.g., HCP(s), is loaded onto an ion exchange column, such as a cation exchange column. For example, but not by way of limitation, the mixture can be loaded at a load of about 80 g protein/L resin depending upon the column used. An example of a suitable cation exchange column is a 80 cm diameter×23 cm long column whose bed volume is about 116 L. The mixture loaded onto this cation column can subsequently washed with wash buffer (equilibration buffer). The antibody is then eluted from the column, and a first eluate is obtained.

This ion exchange step facilitates the capture of the antibody of interest while reducing impurities such as HCPs. In certain aspects, the ion exchange column is a cation exchange column. For example, but not by way of limitation, a suitable resin for such a cation exchange column is CM HyperDF resin. These resins are available from commercial sources such as Pall Corporation. This cation exchange procedure can be carried out at or around room temperature.

4.4 Ultrafiltration/Diafiltration

Certain embodiments of the present invention employ ultrafiltration and/or diafiltration steps to further purify and concentration the anti-IL-12 antibody sample, Ultrafiltration is described in detail in, Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally referred to filtration using filters with a pore size of smaller than 0.1 μm. By employing filters having such small pore size, the volume of the sample can be reduced through permeation of the sample buffer through the filter while the anti-IL-12 antibodies is be retained.

Diafiltration is a method of using ultrafilters to remove and exchange salts, sugars, non-aqueous solvents, separation of free from bound species, removal of material of low molecular weight, or cause the rapid change of ionic and/or pH environments. Such microsolutes are removed most efficiently by adding solvent to the solution being ultrafiltered at a rate equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

4.5 Hydrophobic Interaction Chromatography

The present invention also features methods for producing a HCP-reduced antibody preparation from a mixture comprising an antibody and at least one HCP further comprising a hydrophobic interaction separation step. For example, a first eluate obtained from an ion exchange column can be subjected to a hydrophobic interaction material such that a second eluate having a reduced level of HCP is obtained. Hydrophobic interaction chromatography steps, such as those disclosed herein, are generally performed to remove protein aggregates, such as antibody aggregates, and process-related impurities.

In performing the separation, the sample mixture is contacted with the HIC material, e.g., using a batch purification technique or using a column. Prior to HIC purification it may be desirable to remove any chaotropic agents or very hydrophobic substances, e.g., by passing the mixture through a pre-column.

For example, in the context of batch purification, HIC material is prepared in or equilibrated to the desired equilibration buffer. A slurry of the HIC material is obtained. The antibody solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb antibodies that have bound to the HIC material. In order to elute bound antibodies, the salt concentration can be decreased.

Whereas ion exchange chromatography relies on the charges of the antibodies to isolate them, hydrophobic interaction chromatography uses the hydrophobic properties of the antibodies. Hydrophobic groups on the antibody interact with hydrophobic groups on the column. The more hydrophobic a protein is the stronger it will interact with the column. Thus the HIC step removes host cell derived impurities (e.g., DNA and other high and low molecular weight product-related species).

Hydrophobic interactions are strongest at high ionic strength, therefore, this form of separation is conveniently performed following salt precipitations or ion exchange procedures. Adsorption of the antibody to a HIC column is favored by high salt concentrations, but the actual concentrations can vary over a wide range depending on the nature of the antibody and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺; Ca⁺⁺; Mg⁺⁺; Li⁺; Cs⁺; Na⁺; K⁺; Rb⁺; NH₄⁺, while anions may be ranked in terms of increasing chaotropic effect as P0⁻; S0₄⁻; CH₃CO₃⁻; Cl⁻; Br⁻; NO₃⁻; ClO₄⁻; I⁻; SCN⁻.

In general, Na, K or NH₄ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful.

HIC columns normally comprise a base matrix (e.g., cross-linked agarose or synthetic copolymer material) to which hydrobobic ligands (e.g., alkyl or aryl groups) are coupled. A suitable HIC column comprises an agarose resin substituted with phenyl groups (e.g., a Phenyl Sepharose™ column). Many HIC columns are available commercially. Examples include, but are not limited to, Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Mehyl or Macro-Prep™ t-Butyl Supports (Bio-Rad, Calif.); WP HI-Propyl (C₃)™ column (J. T. Baker, N.J.); and Toyopearl™ ether, phenyl or butyl columns (TosoHaas, Pa.)

4.6 Exemplary Purification Strategies

In certain embodiments, primary recovery can proceed by sequentially employing pH reduction, centrifugation, and filtration steps to remove cells and cell debris (including HCPs) from the production bioreactor harvest. For example, but not by way of limitation, a culture comprising antibodies, media, and cells can be subjected to pH inactivation using a pH of about 3.5 for approximately 1 hour. The pH reduction can be facilitated using known acid preparations such as citric acid, e.g., 3 M citric acid. Such pH reduction reduces and/or inactivates, if not completely eliminates, pH sensitive virus contaminants and precipitates some media/cell contaminants. Following such reduction, the pH is adjusted to about 4.9 or 5.0 using a base such as sodium hydroxide, e.g., 3 M sodium hydroxide, for about twenty to about forty minutes. This adjustment can occur at around 20° C. In certain embodiments, the pH adjusted culture then centrifuged at approximately 7000×g to approximately 11,000×g. In certain embodiments, the resulting sample supernatant is then passed through a filter train comprising multiple depth filters. In certain embodiments, the filter train comprises around twelve 16-inch Cuno™ model 30/60ZA depth filters (3M Corp.) and around three round filter housings fitted with three 30-inch 0.45/0.2 μm Sartopore™ 2 filter cartridges (Sartorius). The clarified supernatant is collected in a vessel such as a pre-sterilized harvest vessel and held at approximately 8° C. This temperature is then adjusted to approximately 20° C. prior to the capture chromatography step or steps outlined below. It should be noted that one skilled in the art may vary the conditions recited above and still be within the scope of the present invention.

The clarified supernatant can then be further purified using a cation exchange column. In certain embodiments, the equilibrating buffer used in the cation exchange column is a buffer having a pH of about 5.0. An example of a suitable buffer is about 210 mM sodium acetate, pH 5.0. Following equilibration, the column is loaded with sample prepared from the primary recovery step above. The column is packed with an cation exchange resin, such as CM Sepharose™ Fast Flow from GE Healthcare. The column is then washed using the equilibrating buffer. The column is next subjected to an elution step using a buffer having a greater ionic strength as compared to the equilibrating or wash buffer. For example, a suitable elution buffer can be about 790 mM sodium acetate, pH 5.0. The anti-IL-12 antibodies will be eluted and can be monitored using a UV spectrophotometer set at OD_{280 nm}. In a particular example, elution collection can be from upside 3 OD_{280 nm} to downside 8 OD_{28 nm}. It should be understood that one skilled in the art may vary the conditions and yet still be within the scope of the invention.

In certain embodiments the clarified supernatant obtained from the primary recovery is instead further purified using an anion exchange column. A non-limiting example of a suitable column for this step is a 60 cm diameter×30 cm long column whose bed volume is about 85 L. The column is packed with an anion exchange resin, such as Q Sepharose™ Fast Flow from GE Healthcare. The column can be equilibrated using about seven column volumes of an appropriate buffer such as Tris/sodium chloride. An example of suitable conditions are 25 mM Tris, 50 mM sodium chloride at pH 8.0. Again, a skill artisan may vary the conditions but still be within the scope of the present invention. The column is loaded with the collected sample from the primary recovery step outlined above. In another aspect, the column is loaded from the eluate collected during cation exchange. Following the loading of the column, the column is washed with the equilibration buffer (e.g., the Tris/sodium chloride buffer). The flow-through comprising the anti-IL-12 antibodies can be monitored using a UV spectrophotometer at OD_{280 nm}. This anion exchange step reduces process related impurities such as nucleic acids like DNA, and host cell proteins. The separation occurs due to the fact that the antibodies of interest do not interact with nor bind to the solid phase of the column, e.g., to the Q Sepharose™, but many impurities do interact with and bind to the column's solid phase. The anion exchange can be performed at about 12° C.

In certain embodiments, the cation exchange or anion exchange eluate, depending on which ion exchange step is employed first, is next filtered using, e.g., a 16 inch Cuno™ delipid filter. This filtration, using the delipid filter, can be followed by, e.g., a 30-inch 0.45/0.2 μm Sartopore™ bi-layer filter cartridge. The ion exchange elution buffer can be used to flush the residual volume remaining in the filters and prepared for ultrafiltration/diafiltration.

In order to accomplish the ultratfiltration/diafiltration step, the filtration media is prepared in a suitable buffer, e.g., 20 mM sodium phosphate, pH 7.0. A salt such as sodium chloride can be added to increase the ionic strength, e.g., 100 mM sodium chloride. This ultrafiltration/diafiltration step serves to concentrate the anti-IL-12 antibodies, remove the sodium acetate and adjust the pH. Commercial filters are available to effectuate this step. For example, Millipore manufactures a 30 kD molecular weight cut-off (MWCO) cellulose ultrafilter membrane cassette. This filtration procedure can be conducted at or around room temperature.

In certain embodiments, the sample from the capture filtration step above is subjected to a second ion exchange separation. Preferably this second ion exchange separation will involve separation based on the opposite charge of the first ion exchange separation. For example, if an anion exchange step is employed after primary recovery, the second ion exchange chromatographic step be a cationic exchange step. Conversely, if the primary recovery step was followed by a cationic exchange step, that step would be followed by an anionic exchange step. In certain embodiments the first ion exchange elute can be subjected directly to the second ion exchange chromatographic step where the first ion exchange elute is adjusted to the appropriate buffer conditions. Suitable anionic and cationic separation materials and conditions are described above.

In certain embodiments of the instant invention the sample containing anti-IL-12 antibodies will be further processed using a hydrophobic interaction separation step. A non-limiting example of a suitable column for such a step is an 80 cm diameter×15 cm long column whose bed volume is about 75 L, which is packed with an appropriate resin used for HIC such as, but not limited to, Phenyl HP Sepharose™ from Amersham Biosciences, Upsala, Sweden. The flow-through preparation obtained from the previous anion exchange chromatography step comprising the antibodies of interest can be diluted with an equal volume of around 1.7 M ammonium sulfate, 50 mM sodium phosphate, pH 7.0. This then can be subjected to filtration using a 0.45/0.2 μm Sartopore™ 2 bi-layer filter, or its equivalent. In certain embodiments, the hydrophobic chromatography procedure involves two or more cycles.

In certain embodiments, the HIC column is first equilibrated using a suitable buffer. A non-limiting example of a suitable buffer is 0.85 M ammonium sulfate, 50 mM sodium phosphate, pH 7.0. One skilled in the art can vary the equilibrating buffer and still be within the scope of the present invention by altering the concentrations of the buffering agents and/or by substituting equivalent buffers. In certain embodiments the column is then loaded with an anion exchange flow-through sample and washed multiple times, e.g., three times, with an appropriate buffer system such as ammonium sulfate/sodium phosphate. An example of a suitable buffer system includes 1.1 M ammonium sulfate, 50 mM sodium phosphate buffer with a pH of around 7.0. Optionally, the column can undergo further wash cycles. For example, a second wash cycle can include multiple column washes, e.g., one to seven times, using an appropriate buffer system. A non-limiting example of a suitable buffer system includes 0.85 M ammonium sulfate, 50 mM sodium phosphate, pH 7.0. In one aspect, the loaded column undergoes yet a third wash using an appropriate buffer system. The column can be washed multiple times, e.g., one to three times, using a buffer system such as 1.1 M ammonium sulfate, 50 mM sodium phosphate at a pH around 7.0. Again, one skilled in the art can vary the buffering conditions and still be within the scope of the present invention

The column is eluted using an appropriate elution buffer. A suitable example of such an elution buffer is 0.5 M ammonium sulfate, 15 mM sodium phosphate at a pH around 7.0. The antibodies of interest can be detected and collected using a conventional spectrophotometer from the upside at 3 OD_{280 nm} to downside of peak at 3 OD_{280 nm}.

In certain aspects of the invention, the eluate from the hydrophobic chromatography step is subjected to filtration for the removal of viral particles, including intact viruses, if present. A non-limiting example of a suitable filter is the Ultipor DV50™ filter from Pall Corporation. Other viral filters can be used in this filtration step and are well known to those skilled in the art. The HIC eluate is passed through a pre-wetted filter of about 0.1 μm and a 2×30-inch Ultipor DV50™ filter train at around 34 psig. In certain embodiments, following the filtration process, the filter is washed using, e.g., the HIC elution buffer in order to remove any antibodies retained in the filter housing. The filtrate can be stored in a pre-sterilized container at around 12° C.

In a certain embodiments, the filtrate from the above is again subjected to ultrafiltration/diafiltration. This step is important if a practitioner's end point is to use the antibody in a, e.g., pharmaceutical formulation. This process, if employed, can facilitate the concentration of antibody, removal of buffering salts previously used and replace it with a particular formulation buffer. In certain embodiments, continuous diafiltration with multiple volumes, e.g., two volumes, of a formulation buffer is performed. A non-limiting example of a suitable formulation buffer is 5 mM methionine, 2% mannitol, 0.5% sucrose, pH 5.9 buffer (no Tween). Upon completion of this diavolume exchange the antibodies are concentrated. Once a predetermined concentration of antibody has been achieved, then a practitioner can calculate the amount of 10% Tween that should be added to arrive at a final Tween concentration of about 0.005% (v/v).

Certain embodiments of the present invention will include further purification steps. Examples of additional purification procedures which can be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, dialysis, and affinity chromatography (e.g., using protein A, protein G, an antibody, a specific substrate, ligand or antigen as the capture reagent).

In certain embodiments of the present invention, the anti-IL-12 antibody is an IgA₁, IgA₂, IgD, IgE, IgG₁, IgG₂, IgG₃, IgG₄, or IgM isotype antibody comprising the heavy and light chain variable region sequences outlined in FIG. 1. In preferred embodiments, the anti-IL-12 antibody is an IgG₁, IgG₂, IgG₃ or IgG₄ isotype antibody comprising the heavy and light chain variable region sequences outlined in FIG. 1, more preferably the anti-IL-12 antibody is an IgG₁ isotype antibody comprising the heavy and light chain variable region sequences outlined in FIG. 1.

### 5. Methods of Assaying Sample Purity

The present invention also provides methods for determining the residual levels of host cell protein (HCP) concentration in the isolated/purified antibody composition. As described above, HCPs are desirably excluded from the final target substance product, the anti-IL-12 antibody. Exemplary HCPs include proteins originating from the source of the antibody production. Failure to identify and sufficiently remove HCPs from the target antibody may lead to reduced efficacy and/or adverse subject reactions.

As used herein, the term “HCP ELISA” refers to an ELISA where the second antibody used in the assay is specific to the HCPs produced from cells, e.g., CHO cells, used to generate the antibody, anti-IL-12 antibody. The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with antibody DNA. In an exemplary embodiment, the second antibody is produced using HPCs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP Biotinylated, is added, and binds to the HCPs within the sample. In certain embodiments the first and second antibodies are polyclonal antibodies. In certain aspects the first and second antibodies are blends of polyclonal antibodies raised against HCPs, for example, but not limited to Biotinylated goat anti Host Cell Protein Mixture 599/626/748. The amount of HCP contained in the sample is determined using the appropriate test based on the label of the second antibody.

HCP ELISA may be used for determining the level of HCPs in an antibody composition, such as an eluate or flow-through obtained using the process described in section III above. The present invention also provides a composition comprising an antibody, wherein the composition has no detectable level of HCPs as determined by an HCP Enzyme Linked Immunosorbent Assay (“ELISA”).

### 6. Further Modifications

The anti-IL-12 antibodies of the present invention can be modified. In some embodiments, the anti-IL-12 antibodies or antigen binding fragments thereof, are chemically modified to provide a desired effect. For example, pegylation of antibodies and antibody fragments of the invention may be carried out by any of the pegylation reactions known in the art, as described, e.g., in the following references: Focus on Growth Factors 3:4-10 (1992); EP 0 154 316; and EP 0 401 384, each of which is incorporated by reference herein in its entirety. In one aspect, the pegylation is carried out via an acylation reaction or an alkylation reaction with a reactive polyethylene glycol molecule (or an analogous reactive water-soluble polymer). A suitable water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl-ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under suitable conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat IL-12-related disorders of the invention by administration of the anti-IL-12 antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

An antibody or antibody portion of the invention can be derivatized or linked to another functional molecule (e.g., another peptide or protein). Accordingly, the antibodies and antibody portions of the invention are intended to include derivatized and otherwise modified forms of the human anti-hIL-12 antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### 7. Pharmaceutical Compositions

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof In many cases, it is desirable to include isotonic agents, e.g., sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody-portions of the invention can be incorporated into a pharmaceutical composition suitable for parenteral administration. The antibody or antibody-portions can be prepared as an injectable solution containing, e.g., 0.1-250 mg/mL antibody. The injectable solution can be composed of either a liquid or lyophilized dosage form in a flint or amber vial, ampule or pre-filled syringe. The buffer can be L-histidine approximately 1-50 mM, (optimally 5-10 mM), at pH 5.0 to 7.0 (optimally pH 6.0). Other suitable buffers include but are not limited to sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage faun, principally 0-10% sucrose (optimally 0.5-1.0%). Other suitable cryoprotectants include trehalose and lactose. Bulking agents can be included for a lyophilized dosage form, principally 1-10% mannitol (optimally 24%). Stabilizers can be used in both liquid and lyophilized dosage forms, principally 1-50 mM L-methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In one aspect, the pharmaceutical composition includes the antibody at a dosage of about 0.01 mg/kg-10 mg/kg. In another aspect, the dosages of the antibody include approximately 1 mg/kg administered every other week, or approximately 0.3 mg/kg administered weekly. A skilled practitioner can ascertain the proper dosage and regime for administering to a subject.

The compositions of this invention may be in a variety of forms. These include, e.g., liquid, semi-solid and solid dosage forms, such as liquid solutions (e.g., injectable and infusible solutions), dispersions or suspensions, tablets, pills, powders, liposomes and suppositories. The form depends on, e.g., the intended mode of administration and therapeutic application. Typical compositions are in the form of injectable or infusible solutions, such as compositions similar to those used for passive immunization of humans with other antibodies. One mode of administration is parenteral (e.g., intravenous, subcutaneous, intraperitoneal, intramuscular). In one aspect, the antibody is administered by intravenous infusion or injection. In another aspect, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof The proper fluidity of a solution can be maintained, e.g., by the use of a coating such as lecithin, by the maintenance of the required particle size in the case of dispersion and by the use of surfactants. Prolonged absorption of injectable compositions can be brought about by including in the composition an agent that delays absorption, e.g., monostearate salts and gelatin.

The antibodies and antibody-portions of the present invention can be administered by a variety of methods known in the art, one route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, the entire teaching of which is incorporated herein by reference.

In certain aspects, an antibody or antibody portion of the invention may be orally administered, e.g., with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain aspects, an antibody or antibody portion of the invention is co-formulated with and/or co-administered with one or more additional therapeutic agents that are useful for treating disorders in which IL-12 activity is detrimental. For example, an anti-hIL-12 antibody or antibody portion of the invention may be co-formulated and/or co-administered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition, e.g., an agent which effects the viscosity of the composition.

It should further be understood that the combinations which are to be included within this invention are those combinations useful for their intended purpose. The agents set forth below are illustrative and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function.

Some combinations are non-steroidal anti-inflammatory drug(s) also referred to as NSAIDS which include drugs like ibuprofen. Other combinations are corticosteroids including prednisolone; the well known side-effects of steroid use can be reduced or even eliminated by tapering the steroid dose required when treating patients in combination with the anti-IL-12 antibodies of this invention. Non-limiting examples of therapeutic agents for rheumatoid arthritis with which an antibody, or antibody portion, of the invention can be combined to include the following: cytokine suppressive anti-inflammatory drug(s) (CSAIDs); antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, or their ligands including CD 154 (gp39 or CD40L).

Some combinations of therapeutic agents may interfere at different points in the autoimmune and subsequent inflammatory cascade; examples include TNF antagonists like chimeric, humanized or human TNF antibodies, D2E7, (U.S. application Ser. No. 08/599,226 filed Feb. 9, 1996, the entire teaching of which is incorporated herein by reference), cA2 (Remicade™), CDP 571, anti-TNF antibody fragments (e.g., CDP870), and soluble p55 or p75 TNF receptors, derivatives thereof, (p75TNFRIgG (Enbrel™) or p55TNFR1gG (Lenercept), soluble IL-13 receptor (sIL-13), and also TNFα converting enzyme (TACE) inhibitors; similarly IL-1 inhibitors (e.g., Interleukin-1-converting enzyme inhibitors, such as Vx740, or IL-1RA, etc.) may be effective for the same reason. Other combinations include Interleukin 11, anti-P7s and p-selectin glycoprotein ligand (PSGL). Yet other combinations involve other key players of the autoimmune response which may act parallel to, dependent on or in concert with IL-12 function; especially included are IL-18 antagonists including IL-18 antibodies or soluble IL-18 receptors, or IL-18 binding proteins. It has been shown that IL-12 and IL-18 have overlapping but distinct functions and a combination of antagonists to both may be most effective. Yet another combination includes non-depleting anti-CD4 inhibitors. Yet other combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), β-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel.TM.)and p55TNFRIgG (Lenercept), sIL-1 RI, sIL-1RII, sIL-6R, soluble IL-13 receptor (sIL-13)) and anti-inflammatory cytokines (e.g., IL-4, IL-10, IL-11, IL-13 and TGFβ). Some combinations include methotrexate or leflunomide and in moderate or severe rheumatoid arthritis cases, cyclosporine.

Non-limiting examples of therapeutic agents for inflammatory bowel disease with which an antibody, or antibody portion, of the invention can be combined include the following: budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1α monoclonal antibodies, anti-IL-6 monoclonal antibodies, growth factors, elastase inhibitors, pyridinyl-imidazole compounds, antibodies to or antagonists of other human cytokines or growth factors, e.g., TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NSAIDs, e.g., ibuprofen, corticosteroids such as prednisolone, phosphodiesterase inhibitors, adenosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1RI, sIL-1RII, sIL-6R, soluble IL-13 receptor (sIL-13)) and anti-inflammatory cytokines (e.g., IL-4, IL-10, IL-11, IL-13 and TGFβ).

Examples of therapeutic agents for Crohn's disease in which an antibody or an antigen binding portion can be combined include the following: TNF antagonists, e.g., anti-TNF antibodies, D2E7 (U.S. application Ser. No. 08/599,226, filed Feb. 9, 1996, the entire teaching of which is incorporated herein by reference), cA2 (Remicade™), CDP 571, anti-TNF antibody fragments (e.g., CDP870), TNFR-Ig constructs(p75TNFRIgG (Enbrel™) and p55TNFRIgG (Lenercept)), anti-P7s, p-selectin glycoprotein ligand (PSGL), soluble IL-13 receptor (sIL-13), and PDE4 inhibitors. Antibodies of the invention or antigen binding portions thereof, can be combined with corticosteroids, e.g., budenoside and dexamethasone. Antibodies of the invention or antigen binding portions thereof, may also be combined with agents such as sulfasalazine, 5-aminosalicylic acid and olsalazine, and agents which interfere with synthesis or action of proinflammatory cytokines such as IL-1, e.g., IL-1 converting enzyme inhibitors (e.g., Vx740) and IL-1ra. Antibodies of the invention or antigen binding portion thereof may also be used with T cell signaling inhibitors, e.g., tyrosine kinase inhibitors 6-mercaptopurines. Antibodies of the invention or antigen binding portions thereof, can be combined with IL-11.

Non-limiting examples of therapeutic agents for multiple sclerosis with which an antibody, or antibody portion, of the invention can be combined include the following: corticosteroids, prednisolone, methylprednisolone, azathioprine, cyclophosphamide, cyclosporine, methotrexate, 4-aminopyridine, tizanidine, IFNβ1a (Avonex; Biogen), IFNβ1b (Betaseron; Chiron/Berlex), Copolymer 1 (Cop-1, Copaxone, Teva Pharmaceutical Industries, Inc.), hyperbaric oxygen, intravenous immunoglobulin, clabribine, antibodies to or antagonists of other human cytokines or growth factors, e.g., TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80, CD86, CD90 or their ligands. The antibodies of the invention, or antigen binding portions thereof, may also be combined with agents, such as methotrexate, cyclosporine, FK506, rapamycin, mycophenolate mofetil, leflunomide, NSAIDs, e.g., ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1 RI, sIL-1 RII, sIL-6R, soluble IL-13 receptor (sIL-13)) and anti-inflammatory cytokines (e.g., IL-4, IL-10, IL-13 and TGFβ).

Examples of therapeutic agents for multiple sclerosis in which the antibody or antigen binding portion thereof can be combined to include IFNβ, e.g., IFNβ1a and IFNβ1b, copaxone, corticosteroids, IL-1 inhibitors, TNF inhibitors, and antibodies to CD40 ligand and CD80.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments it is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.01-20 mg/kg, or 1-10 mg/kg, or 0.3-1 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### 8. Uses of the Antibodies of the Invention

8.1. Uses Generally

Given their ability to bind to IL-12, the anti-IL-12 antibodies, or portions thereof, of the invention can be used to detect IL-12, in one aspect, hIL-12 (e.g., in a sample matrix, in one aspect, a biological sample, such as serum or plasma), using a conventional immunoassay, such as an enzyme linked immunosorbent assays (ELISA), an radioimmunoassay (RIA) or tissue immunohistochemistry. The invention provides a method for detecting IL-12 in a biological sample comprising contacting a sample with an antibody, or antibody portion, of the invention and detecting either the antibody (or antibody portion) bound to IL-12 or unbound antibody (or antibody portion), to thereby detect IL-12 in the sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S, or ³H. Detection of IL-12 in a sample may be useful in a diagnostic example in the diagnosis of a condition associated with increased IL-12, and/or may be useful in identifying a subject who may benefit from treatment with an anti-IL-12 antibody.

Alternative to labeling the antibody, IL-12 can be assayed in a sample by a competition immunoassay utilizing, e.g., rhIL-12 standards labeled with a detectable substance and an unlabeled anti-IL-12 antibody, such as an anti-hIL-12 antibody. In this assay, the sample, the labeled rhIL-12 standards, and the anti-hIL-12 antibody are combined and the amount of labeled rhIL-12 standard bound to the unlabeled antibody is determined. The amount of hIL-12 in the sample is inversely proportional to the amount of labeled rhIL-12 standard bound to the anti-hIL-12 antibody.

The antibodies and antibody portions of the invention are capable of neutralizing IL-12 activity in vitro and in vivo, in one aspect, a hIL-12 activity. Accordingly, the antibodies and antibody portions of the invention can be used to inhibit IL-12 activity, e.g., in a cell culture containing IL-12, in human subjects or in other mammalian subjects having IL-12 with which an antibody of the invention cross-reacts (e.g., primates such as baboon, cynomolgus and rhesus). In a one aspect, the invention provides an isolated human antibody, or antigen-binding portion thereof, that neutralizes the activity of human IL-12, and at least one additional primate IL-12 selected from the group consisting of baboon IL-12, marmoset IL-12, chimpanzee IL-12, cynomolgus IL-12 and rhesus IL-12, but which does not neutralize the activity of the mouse IL-12. In one aspect, the IL-12 is human IL-12. For example, in a cell culture containing, or suspected of containing hIL-12, an antibody or antibody portion of the invention can be added to the culture medium to inhibit hIL-12 activity in the culture.

In another aspect, the invention provides a method for inhibiting IL-12 activity in a subject suffering from a disorder in which IL-12 activity is detrimental. IL-12 has been implicated in the pathophysiology of a wide variety of disorders (Windhagen et al., (1995) J. Exp. Med. 182: 1985-1996; Morita et al. (1998) Arthritis and Rheumatism. 41: 306-314; Bucht et al., (1996) Clin. Exp. Immunol. 103: 347-367; Fais et al. (1994) J. Interferon Res. 14:235-238; Pyrronchi et al., (1997) Am. J. Path. 150:823-832; Monteleone et al., (1997) Gastroenterology. 112:1169-1178, and Berrebi et al., (1998) Am. J. Path 152:667-672; Pyrronchi et al. (1997) Am. J. Path. 150:823-832, the entire teachings of which are incorporated herein by reference). The invention provides methods for inhibiting IL-12 activity in a subject suffering from such a disorder, which method comprises administering to the subject an antibody or antibody portion of the invention such that IL-12 activity in the subject is inhibited. In one aspect, the IL-12 is human IL-12 and the subject is a human subject. Alternatively, the subject can be a mammal expressing IL-12 with which an antibody of the invention cross-reacts. Still further the subject can be a mammal into which has been introduced hIL-12 (e.g., by administration of hIL-12 or by expression of an hIL-12 transgene). An antibody of the invention can be administered to a human subject for therapeutic purposes. Moreover, an antibody of the invention can be administered to a non-human mammal expressing a IL-12 with which the antibody cross-reacts for veterinary purposes or as an animal model of human disease. Regarding the latter, such animal models may be useful for evaluating the therapeutic efficacy of antibodies of the invention (e.g., testing of dosages and time courses of administration).

As used herein, the phrase “a disorder in which IL-12 activity is detrimental” is intended to include diseases and other disorders in which the presence of IL-12 in a subject suffering from the disorder has been shown to be or is suspected of being either responsible for the pathophysiology of the disorder or a factor that contributes to a worsening of the disorder. Accordingly, a disorder in which IL-12 activity is detrimental is a disorder in which inhibition of IL-12 activity is expected to alleviate the symptoms and/or progression of the disorder. Such disorders may be evidenced, e.g., by an increase in the concentration of IL-12 in a biological fluid of a subject suffering from the disorder (e.g., an increase in the concentration of IL-12 in serum, plasma, synovial fluid, etc. of the subject), which can be detected, e.g., using an anti-IL-12 antibody as described above. There are numerous examples of disorders in which IL-12 activity is detrimental. In one aspect, the antibodies or antigen binding portions thereof, can be used in therapy to treat the diseases or disorders described herein. In another aspect, the antibodies or antigen binding portions thereof, can be used for the manufacture of a medicine for treating the diseases or disorders described herein. The use of the antibodies and antibody portions of the invention in the treatment of a few non-limiting specific disorders is discussed further below.

Interleukin 12 plays a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anemia, Coombs positive haemolytic anaemia, acquired pernicious anemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, and autoimmune uveitis.

In certain aspects, the antibodies of the invention or antigen-binding portions thereof, are used to treat rheumatoid arthritis, Crohn's disease, multiple sclerosis, insulin dependent diabetes mellitus and psoriasis.

8.2 Use in Rheumatoid Arthritis

Interleukin-12 has been implicated in playing a role in inflammatory diseases such as rheumatoid arthritis. Inducible IL-12p40 message has been detected in synovia from rheumatoid arthritis patients and IL-12 has been shown to be present in the synovial fluids from patients with rheumatoid arthritis (see, e.g., Morita et al., (1998) Arthritis and Rheumatism 41: 306-314, the entire teaching of which is incorporated herein by reference). IL-12 positive cells have been found to be present in the sublining layer of the rheumatoid arthritis synovium. The human antibodies, and antibody portions of the invention can be used to treat, e.g., rheumatoid arthritis, juvenile rheumatoid arthritis, Lyme arthritis, rheumatoid spondylitis, osteoarthritis and gouty arthritis. Typically, the antibody, or antibody portion, is administered systemically, although for certain disorders, local administration of the antibody or antibody portion may be beneficial. An antibody, or antibody portion, of the invention also can be administered with one or more additional therapeutic agents useful in the treatment of autoimmune diseases.

In the collagen induced arthritis (CIA) murine model for rheumatoid arthritis, treatment of mice with an anti-IL-12 mAb (rat anti-mouse IL-12 monoclonal antibody, C17.15) prior to arthritis profoundly suppressed the onset, and reduced the incidence and severity of disease. Treatment with the anti-IL-12 mAb early after onset of arthritis reduced severity, but later treatment of the mice with the anti-IL-12 mAb after the onset of disease had minimal effect on disease severity.

8.3 Use in Crohn's Disease

Interleukin-12 also plays a role in the inflammatory bowel disease, Crohn's disease. Increased expression of IFN-γ and IL-12 occurs in the intestinal mucosa of patients with Crohn's disease (see, e.g., Fais et al., (1994) J. Interferon Res. 14: 235-238; Pyrronchi et al., (1997) Amer. J. Pathol. 150: 823-832; Monteleone et al., (1997) Gastroenterology 112: 1169-1178; Berrebi et al., (1998) Amer. J. Pathol. 152: 667-672, the entire teachings of which are incorporated herein by reference). Anti-IL-12 antibodies have been shown to suppress disease in mouse models of colitis, e.g., TNBS induced colitis IL-2 knockout mice, and recently in IL-10 knock-out mice. Accordingly, the antibodies, and antibody portions, of the invention, can be used in the treatment of inflammatory bowel diseases.

8.4 Use in Multiple Sclerosis

Interleukin-12 has been implicated as a key mediator of multiple sclerosis. Expression of the inducible IL-12 p40 message or IL-12 itself can be demonstrated in lesions of patients with multiple sclerosis (Windhagen et al., (1995) J. Exp. Med 182: 1985-1996, Drulovic et al., (1997) J. Neurol. Sci. 147:145-150, the entire teachings of which are incorporated herein by reference). Chronic progressive patients with multiple sclerosis have elevated circulating levels of IL-12. Investigations with T-cells and antigen presenting cells (APCs) from patients with multiple sclerosis revealed a self-perpetuating series of immune interactions as the basis of progressive multiple sclerosis leading to a Th1-type immune response. Increased secretion of IFN-γ from the T cells led to increased IL-12 production by APCs, which perpetuated the cycle leading to a chronic state of a Th1-type immune activation and disease (Balashov et al., (1997) Proc. Natl. Acad. Sci. 94: 599-603, the entire teaching of which is incorporated herein by reference). The role of IL-12 in multiple sclerosis has been investigated using mouse and rat experimental allergic encephalomyelitis (EAE) models of multiple sclerosis. In a relapsing-remitting EAE model of multiple sclerosis in mice, pretreatment with anti-IL-12 mAb delayed paralysis and reduced clinical scores. Treatment with anti-IL-12 mAb at the peak of paralysis or during the subsequent remission period reduced clinical scores. Accordingly, the antibodies or antigen binding portions thereof of the invention nay serve to alleviate symptoms associated with multiple sclerosis in humans.

8.5 Use in Insulin-Dependent Diabetes Mellitus

Interleukin-12 has been implicated as an important mediator of insulin-dependent diabetes mellitus (IDDM). IDDM was induced in NOD mice by administration of IL-12, and anti-IL-12 antibodies were protective in an adoptive transfer model of IDDM. Early onset IDDM patients often experience a so-called “honeymoon period” during which some residual islet cell function is maintained. These residual islet cells produce insulin and regulate blood glucose levels better than administered insulin. Treatment of these early onset patients with an anti-IL-12 antibody may prevent further destruction of islet cells, thereby maintaining an endogenous source of insulin.

8.6 Use in Psoriasis

Interleukin-12 has been implicated as a key mediator in psoriasis. Psoriasis involves acute and chronic skin lesions that are associated with a TH 1-type cytokine expression profile. (Hamid et al. (1996) J. Allergy Clin. Immunol. 1:225-231; Turka et al. (1995) Mol. Med. 1:690-699, the entire teachings of which are incorporated herein by reference). IL-12 p35 and p40 mRNAs were detected in diseased human skin samples. Accordingly, the antibodies or antigen binding portions thereof of the invention may serve to alleviate chronic skin disorders such psoriasis.

### Examples

### 1. The Isolation/Purification of Anti-IL-12 Antibodies

This example provides one scheme of purifying anti-IL-12 antibodies from host cell proteins (HCP) as well as from other impurities.

Primary Recovery

Primary recovery by pH reduction, centrifugation and filtration was used to remove cells and cell debris from a production bioreactor harvest. The culture comprising the antibodies of interest, media, and cells was pH inactivated to 3.5 for 1 hour to kill possible pH sensitive virus contaminates and to precipitate media/cell contaminates in the 3000 L production bioreactor. The culture was then adjusted up to pH 4.9. The pH reduction was achieved with 3 M citric acid over the course of 20 to 40 minutes. The pH increase was performed using 3 M sodium hydroxide over the course of 20 to 40 minutes. These operations occured at a temperature of 20° C. Post pH inactivation the culture was centrifuged into another bioreactor used as a holding tank. The centrifuge was run at 11,000×g at a feed rate of 28 L/min. The discharge interval volume was set at 300 seconds to achieve a low turbidity level of 150. The centrifuge filtrate was passed through a filter train comprising twelve 16-inch Cuno™ model 30/60ZA depth filters and a three round filter housing fitted with three 30-inch 0.45/0.2 μm Sartopore™ filter cartridges. The clarified supernatant was collected in a pre-sterilized 3000 L fixed harvest tank and held at 8° C. The temperature was adjusted up to 20° C. prior to ion exchange chromatography.

Titers of ABT-874 at harvest, for various samples designated 28085BI, 28204BI, 28206BI, 28207BI, and 34142BI, ranged from 3.76 to 4.05 mg/mL with an average of 3.91 mg/mL. The pH reduction hold of the cell culture broth and subsequent pH increase and centrifugation of harvest resulted in mass recovery yields ranging from 77% to 84% with an average of 82% and a standard deviation of 2.77 (see Tables 2 and 3). Antibody quantitation was determined using a Poros A™ quantitation assay (well known to those skilled in the art) throughout this step.

TABLE 2
Primary recovery data of various sample lots
Primary Recovery

| | Fermentation lot # | | | | |
|---|---|---|---|---|---|
| Process step | 28085BI | 28204BI | 28206BI | 28207BI | 34142BI |
| Ab (product) concentration at harvest | 3.9 | 4.05 | 3.84 | 3.99 | 3.76 |
| (g/L) | | | | | |
| Final culture weight (Kg) | 2439 | 2387 | 2467 | 2494 | 2464 |
| Total product at harvest (g) | 9512 | 9667 | 9473 | 9951 | 9265 |
| 3 M citric acid added (g) | 78 | 62 | 79 | 95 | 81 |
| pH during reduction | 3.5 | 3.5 | 3.5 | 3.5 | 3.5 |
| Duration of low pH (minutes) | 63 | 70 | 60 | 69 | 64 |
| 3 M Sodium hydroxide added (g) | 111 | 89 | 117 | 142 | 116 |
| pH post pH reduction | 4.89 | 4.9 | 4.9 | 4.9 | 4.85 |
| Final clarified harvest weight (Kg) | 2478 | 2402 | 2531 | 2447 | 2449 |
| Product clarified harvest | 3.23 | 3.33 | 3.09 | 3.15 | 3.11 |
| concentration (g/L) | | | | | |
| Total product clarified harvest (g) | 8004 | 7997 | 7821 | 7708 | 7616 |
| Overall harvest product step yield (%) | 84 | 83 | 83 | 77 | 82 |

TABLE 3
Analysis of the primary recovery step

| | Primary recovery averages | | |
|---|---|---|---|
| | Five sample lots | | |
| Process step | *AVG | *SD | *% CV |
| Product concentration at harvest (g/L) | 3.91 | 0.116 | 2.97 |
| Final culture weight (Kg) | 2450 | 40.3 | 1.64 |
| Total product at harvest (g) | 9574 | 255.1 | 2.66 |
| Final clarified harvest weight (Kg) | 2461 | 47.6 | 1.93 |
| Product clarified harvest concentration (g/L) | 3.18 | 0.1 | 3.14 |
| Total product clarified harvest (g) | 7829 | 172.4 | 2.2 |
| Overall harvest product step yield (%) | 82 | 2.77 | 3.38 |

*AVE = average; SD = standard deviation; % CV = Percent coefficient of variation

Cation Exchange

The objective of the cation exchange capture chromatography step is the capture of antibody from depth filtrate and the reduction of process-related impurities (e.g., host cell proteins, related antibody low molecular weight species and medium components). CM HyperDF™ resin (Pall Corporation) was utilized for this process step. The CM HyperDF™ capture step was performed at ambient temperature.

A 80 cm diameter×23 cm long column (bed volume 116 L) was used for this operation. Pre-equilibration, equilibration, load, wash, and regeneration column steps were performed at 16.0 L/min (linear velocity=191 cm/hr). The elution, strip, wash, neutralization, sanitize, and store column steps were performed at 9.2 L/min (linear velocity=110 cm/hr). The column was equilibrated with 210 mM sodium acetate, pH 5.0. Following equilibration, the column was loaded with clarified harvest that is diluted in-line with USP purified water. The column was loaded at a maximum of 80 g of protein per liter of resin (≦9248 g per cycle). The column was then washed to baseline with equilibration buffer. The product was eluted with 790 mM sodium acetate, pH 5.0. Elution collection was from upside at 3 OD_{280 nm} to downside 8 OD_{280 nm} of the antibody elution peak.

The column was regenerated with 1 M sodium chloride, washed with lipid wash 1 M acetic acid/20% isopropyl alcohol, washed with 790 mM sodium acetate, pH 5.0, sanitized with 0.5 M sodium hydroxide, followed with 790 mM sodium acetate, pH 5.0, and stored with 25 mM sodium phosphate, 20% isopropyl alcohol (IPA).

One cycle of the CM HyperDF™ chromatography step was performed for each sample lot examined. The average load for the sample lots was 67.38 g antibody/L of CM HyperDF™ resin with standard deviation of 1.37; ranging from 65.27 to 68.62 g/L (see Tables 4 and 5). The product recovery yields ranged from 83% to 96% with an average of 88% for lots 29001BF, 29008BF, 30005BF, 30006BF, and 35036BF and standard deviation of 5.17. For the clarified harvest, antibody quantitation was determined using a Poros A™ quantitation assay and for the CM HyperDF™ eluate, A_{280 nm} quantitation was used. The cutting criteria for the downside of the CM HyperDF™ elution product peak was high to cut the double light chain product related antibody species to achieve product purity. This made the overall CM HyperDF™ chromatography step yields go from around 95% to mid 80%.

No significant differences in purity by size exclusion (SE)-HPLC were noted for the five cycles. Table 16 shows the average of 94.76% for monomeric IgG with a standard deviation of 0.67.

TABLE 4
CM HyperDF ™ chromatography data
CM HyperDF ™ chromatography

| | Sample lot # | | | | |
|---|---|---|---|---|---|
| Process step | 29001BF | 29008BF | 30005BF | 30006BF | 35036BF |
| Load volume of clarified harvest (Kg = | 2464 | 2380 | 2512 | 2418 | 2434 |
| L) | | | | | |
| Concentration of product load on | 3.23 | 3.33 | 3.09 | 3.15 | 3.11 |
| CM HyperDF ™ (clarified harvest) | | | | | |
| (g/L) | | | | | |
| Total product of load on CM | 7959 | 7926 | 7761 | 7615 | 7571 |
| HyperDF ™ (g) | | | | | |
| Product loading capacity per CM | 68.62 | 68.33 | 66.90 | 65.65 | 65.27 |
| HyperDF ™ resin (g/L) | | | | | |
| Eluate peak start volume (L) from | 89.8 | 88.5 | 64.9 | 72.9 | 58.6 |
| program | | | | | |
| Eluate peak end volume (L) from | 288.1 | 286 | 306.1 | 295.0 | 329.6 |
| program | | | | | |
| Eluate volume of CM HyperDF ™ (Kg = | 200.8 | 199.1 | 245.2 | 227.7 | 275.8 |
| L) | | | | | |
| Eluate concentration of CM | 33.05 | 35.6 | 26.72 | 31.97 | 24.39 |
| HyperDF ™ (g/L) | | | | | |
| Total product of CM HyperDF ™ | 6636 | 7088 | 6552 | 7280 | 6727 |
| eluate (g) | | | | | |
| Overall product yield for step(%) | 83 | 89 | 84 | 96 | 89 |

TABLE 5
Analysis of the CM HyperDF ™ chromatography step

| | CM HyperDF ™ chromatography averages | | |
|---|---|---|---|
| | Five sample lots | | |
| Process step | AVG | SD | % CV |
| Load volume of clarified harvest (Kg = L) | 2442 | 49.5 | 2.03 |
| Concentration of product load on CM HyperDF ™ | 3.18 | 0.099 | 3.11 |
| (clarified harvest) (g/L) | | | |
| Total product of load on CM HyperDF ™ (g) | 7767 | 175.9 | 2.26 |
| Product loading capacity per CM HyperDF ™ resin | 67.38 | 1.374 | 2.04 |
| (g/L) | | | |
| Eluate peak start volume (L) | 74.9 | 13.93 | 18.6 |
| Eluate peak end volume (L) | 301 | 17.83 | 5.92 |
| Eluate volume of CM HyperDF ™ (Kg = L) | 229.7 | 32.18 | 14.01 |
| Eluate concentration of CM HyperDF ™ (g/L) | 30.35 | 4.642 | 15.29 |
| Total product of CM HyperDF ™ eluate (g) | 6857 | 312.6 | 4.56 |
| Overall product yield for step (%) | 88 | 5.17 | 5.88 |

Capture Ultrafiltration/Diafiltration (UF/DF)

The CM HyperDF™ eluate was filtered via delipid depth filter (1×16-inch, Cuno™ Corporation) followed with a 0.45/0.2 μm Sartopore™ bi-layer filter cartridge (1×30-inch). CM HyperDF™ eluate buffer 790 mM sodium acetate, pH 5.0 was used to flush the residual volume left in the filters. Ultrafiltration/difiltration (UF/DF) of the delipid CM HyperDF™ eluate was performed to concentrate the antibodies of interest, remove sodium acetate and buffer exchange of product in 100 mM sodium chloride, 20 mM sodium phosphate, pH 7.0. Millipore Corporation regenerated cellulose ultrafilter type membrane cassettes with a 30 kD molecular weight cut-off (MWCO) were used for this step at ambient temperature.

The 30 kD regenerated cellulose membrane was flushed with 790 mL sodium acetate, pH 5.0 before the start of the addition of product. The delipid-CM HyperDF™ eluate was concentrated to 40 g/L protein with inlet pressures of 20-30 psig and outlet pressures of 10-15 psig and retentate flow rate from 52 L/min to 104 L/min. Continuous diafiltration with a minimum of six volumes of 100 mM sodium chloride, 20 mM sodium phosphate, pH 7.0 was performed. The UF system was then drained of product at a target concentration of 42.5 g/L protein. The system was rinsed with diafiltration buffer to recover product held up in the system. The concentrate and wash were combined to produce the diafiltered ABT-874. This step was performed at ambient temperature. The sample matrix was filtered with an OpticapXLT30™ capsule 2.0/1.2 μm filter followed with a 0.45/0.2 μm Sartopore™ bi-layer filter cartridge (1×30-inch).

The delipid filtration and UF/DF of CM HyperDF™ eluate step resulted in an average product recovery yield of 5.92 Kg for the various sample lots (i.e. , 29001BF, 29008BF, 30005BF, 30006BF, and 35036BF); ranging from 80%-94% (see Tables 6 and 7).

Purity by SE-HPLC were acceptable for the five cycles. Table 16 shows the average of 95.79% for the IgG with a standard deviation of 0.57.

TABLE 6
Delipid filtering and UF/DF of CM HyperDF eluate data of sample lots
Delipid filtering and UF/DF of CM HyperDF ™ eluate

| | Purification sample lot # | | | | |
|---|---|---|---|---|---|
| Process step | 29001BF | 29008BF | 30005BF | 30006BF | 35036BF |
| Start volume of CM HyperDF ™ | 200.8 | 199.1 | 245.2 | 227.7 | 275.8 |
| eluate (Kg = L) | | | | | |
| Eluate concentration of CM | 33.05 | 35.6 | 26.72 | 31.97 | 24.39 |
| HyperDF ™ (g/L) | | | | | |
| Total product of CM HyperDF ™ | 6636 | 7088 | 6552 | 7280 | 6727 |
| eluate (g) | | | | | |
| Volume CM eluate buffer to flush | 70 | 71 | 135 | 143 | 141 |
| delipid filter (L) | | | | | |
| Volume of diafiltration buffer used | 1003 | 1106 | 913 | 1034 | 1051 |
| (L) | | | | | |
| UF/DF retentate volume (Kg = L) | 136.8 | 163.6 | 149.9 | 189.8 | 163.6 |
| UF/DF retentate concentration (g/L) | 38.93 | 36.47 | 40.36 | 31.18 | 38.72 |
| Total product of UF/DF retentate (g) | 5326 | 5966 | 6050 | 5918 | 6334 |
| Overall product yield for step (%) | 80 | 84 | 92 | 81 | 94 |

TABLE 7
Analysis delipid filtering and UF/DF of CM HyperDF ™ eluate data

| | Delipid filtering and UF/DF averages | | |
|---|---|---|---|
| | Five sample lots | | |
| Process step | AVG | SD | % CV |
| Start volume of CM HyperDF ™ eluate (Kg = L) | 229.72 | 32.175 | 14.01 |
| Eluate concentration of CM HyperDF ™ (g/L) | 30.35 | 4.642 | 15.29 |
| Total product of CM HyperDF ™ eluate (g) | 6857 | 312.6 | 4.56 |
| Volume CM eluate buffer to flush delipid filter (L) | 112 | 38 | 33.93 |
| Volume of diafiltration buffer used (L) | 1021 | 71.1 | 6.96 |
| UF/DF retentate volume (Kg = L) | 160.7 | 19.694 | 12.26 |
| UF/DF retentate concentration (g/L) | 37.13 | 3.607 | 9.71 |
| Total product of UF/DF retentate (g) | 5919 | 368.9 | 6.23 |
| Overall product yield for step (%) | 86 | 6.42 | 7.47 |

Anion Exchange Chromatography

Anion exchange chromatography reduces process-related impurities such as DNA and host cell proteins. This process step is a flow through mode chromatography where the main antibody product does not bind to the Q Sepharose™, but the impurities do. This and subsequent steps were performed in a Class 10,000 purification suite at 12±2° C. after the capture UF/DF intermediate product was transferred to the fine purification suite in a mobile, closed tank.

A 60 cm diameter×30 cm long column (bed volume 85 L) was used. The column was packed with Q Sepharose™ Fast Flow anion exchange chromatography resin (Amersham Biosciences, Uppsala, Sweden). All column steps were performed at 7.0 L/min (linear velocity=150 cm/hr), except for storage of the column which is run at 3.5 L/min.

The column was equilibrated using seven column volumes (CVs) of 50 mM sodium chloride, 25 mM Tris, pH 8.0. The maximum protein loading for this step was 50 g of protein per L of resin 4250 g per cycle). This column was cycled twice with only a regeneration of 1 M sodium chloride between cycles. Capture UF/DF material was diluted approximately two fold with 25 mM Tris, pH 8.0. This became the Q load at about 7.0 mS/cm, pH 7.5 to 8.1. After loading of the diluted capture UF/DF material, the column was washed with equilibration buffer. Flow-through comprising the antibodies of interest was collected from the upside at 3 OD_{280 nm} to downside of peak at 3 OD_{280 nm} including the wash after the load. This was the Q flow-through plus wash (Q FTW).

After the second cycle, the column was regenerated with 1 M sodium chloride, washed with Water For Injection (WFI), sanitized for 1 hour with 1 M sodium hydroxide, washed with 1 M sodium chloride, 25 mM sodium phosphate, pH 7.0, to bring the pH down and then stored with 25 mM sodium phosphate, 20% IPA.

Two cycles of the Q Sepharose™ column were performed for each of the sample lots produced. The average load per cycle were 34.8 and 31 g/L resin (cycle A and cycle B, respectively); ranging from 29.01 to 37.13 g/L (see Tables 8 and 9). The overall step yield including both cycles was 92% with a standard deviation of 17.8. This step typically yields 99 to 101% (sample lots 29001BF, 29008BF, 30005BF, and 30006BF). The Q Sepharose™ FTW overall yield was 25.81 kg for the five batches. Q load pH was pH 7.5 with conductivities about 6.0 to 6.7 mS/cm for lot 30006BF.

Purity by SE-HPLC for the five cycles shows an average of 96.75% for the IgG with a standard deviation of 0.53 (Table 16).

TABLE 8
Q Sepharose ™ FF chromatography data of sample lots
Q Sepharose ™ FF chromatography

| | Purification lot # | | | | |
|---|---|---|---|---|---|
| Process step | 29001BF | 29008BF | 30005BF | 30006BF | 35036BF |
| UF/DF retentate volume (Kg = L) undiluted | 136.8 | 163.6 | 149.9 | 189.8 | 163.6 |
| Q load | | | | | |
| UF/DF retentate concentration (g/L) | 38.93 | 36.47 | 40.36 | 31.18 | 38.72 |
| Total product of UF/DF retentate (g) | 5326 | 5966 | 6050 | 5918 | 6335 |
| 25 mM Tris, pH 8 added to dilute UF/DF | 186.8 | 223.6 | 190 | 285.7 | 204 |
| retentate (Kg = L) | | | | | |
| Total volume Q load (L) | 323.6 | 387.2 | 339.9 | 475.5 | 367.6 |
| pH of Q load | 7.5 | 7.5 | 7.5 | 7.5 | 7.5 |
| Conductivity of Q load (mS/cm) | 6.3 | 6.3 | 6.0 | 6.7 | 6.2 |
| Q load concentration (g/L) | 16.46 | 15.4 | 17.8 | 12.4 | 17.2 |
| Cycle A | | | | | |
| Product loaded on column, cycle A (g) | 2667 | 2978 | 3028 | 2945 | 3156 |
| Volume load, cycle A (L) | 162 | 193.4 | 170.1 | 237.5 | 183.5 |
| Actual loading capacity of product/L of | 31.4 | 35.04 | 35.62 | 34.65 | 37.13 |
| Q resin, cycle A (g. product/L. Q resin) | | | | | |
| Volume flow through collected, cycle A (L) | 117.6 | 151.7 | 129.9 | 192 | 136.8 |
| Volume wash collected, cycle A (L) | 76 | 78.9 | 79.3 | 76.9 | 73.6 |
| Volume flow through + wash, cycle A ((Kg = L) | 193.6 | 230.6 | 209.2 | 268.9 | 210.4 |
| Cycle B | | | | | |
| Product loaded on column, cycle B (g) | 2647 | 2894 | 2569 | 2754 | 2466 |
| Volume load, cycle B (L) | 113 | 187.9 | 100 | 222.1 | 143.4 |
| Actual loading capacity of product/L of Q | 31.06 | 34.05 | 30.22 | 32.40 | 29.01 |
| resin, cycle B (g. product/L. Q resin) | | | | | |
| Volume flow through collected, cycle B (L) | 115.7 | 146.4 | 109.7 | 179.7 | 94.8 |
| Volume wash collected, cycle B (L) | 75.7 | 81 | 78.3 | 76.1 | 72.2 |
| Volume flow through + Wash, cycle B ((Kg = L) | 191.4 | 227.4 | 188 | 255.8 | 167 |
| Cycle A + cycle B | | | | | |
| Load cycle A + cycle B (g) | 5314 | 5872 | 5597 | 5699 | 5622 |
| Q flow through + wash volume (Kg = L) | 385 | 458 | 397.2 | 524.7 | 377.4 |
| Q flow through + wash concentration (g/L) | 13.81 | 12.74 | 13.92 | 10.95 | 8.97 |
| Total product of Q flow through + wash (g) | 5317 | 5835 | 5529 | 5745 | 3385 |
| Overall Product Yield for step(%) | 100 | 99 | 99 | 101 | 60 |

TABLE 9
Analysis Q Sepharose ™ FF chromatography data

| | Q Sepharose ™ FF chromatography averages | | |
|---|---|---|---|
| | Five sample lots | | |
| Process step | AVG | SD | % CV |
| UF/DF retentate volume (Kg = L) undiluted Q load | 160.74 | 19.7 | 12.26 |
| UF/DF retentate concentration (g/L) | 37.13 | 3.607 | 9.71 |
| Total product of UF/DF retentate (g) | 5919 | 368.9 | 6.23 |
| 25 mM Tris, pH 8 added to dilute UF/DF retentate | 218 | 40.52 | 18.59 |
| (Kg = L) | | | |
| Total volume Q load (L) | 379 | 59.39 | 15.67 |
| pH of Q load | 7.5 | 0 | 0 |
| Q load concentration (g/L) | 15.9 | 2.13 | 13.4 |
| Cycle A | | | |
| Product loaded on column, cycle A (g) | 2955 | 179.8 | 6.08 |
| Volume load, cycle A (L) | 189 | 29.53 | 15.62 |
| Actual loading of product/L of Q resin, cycle A | 34.80 | 2.11 | 6.1 |
| (g product/L Q resin) | | | |
| Volume flow through collected, cycle A (L) | 146 | 28.71 | 19.66 |
| Volume wash collected, cycle A (L) | 77 | 2.32 | 3.01 |
| Volume flow through + wash, cycle A (Kg = L) | 223 | 29.06 | 13.03 |
| Cycle B | | | |
| Product loaded on column, cycle B (g) | 2666 | 165.5 | 6.21 |
| Volume load, cycle B (L) | 153 | 51.22 | 33.48 |
| Actual loading of product/L of Q resin, cycle B | 31.00 | 1.95 | 6.29 |
| (g product/L Q resin) | | | |
| Volume flow through collected, cycle B (L) | 129 | 33.8 | 26.26 |
| Volume wash collected, cycle B (L) | 77 | 3.27 | 4.25 |
| Volume flow through + wash, cycle B (Kg = L) | 206 | 35.34 | 17.16 |
| Cycle A + cycle B | | | |
| Load cycle A + cycle B (g) | 5621 | 202.4 | 3.6 |
| Q flow through + wash volume (Kg = L) | 428 | 62.47 | 14.6 |
| Q flow through + wash concentration (g/L) | 12.10 | 2.11 | 17.4 |
| Total product of Q flow through + Wash (g) | 5162 | 1014 | 19.63 |
| Overall product yield for step(%) | 92 | 17.8 | 19.35 |

HIC Chromatography

Hydrophobic interaction chromatography (HIC) is used for the removal of antibody aggregates and process-related impurities to final product specifications. This step is a bind and elute mode of chromatography. The anion exchange product (sample) was put in a high salt buffer (ammonium sulfate), bound to the column, and eluted from the column after three washes.

An 80 cm diameter×15 cm long column (bed volume 75 L) was used for this procedure. The column was packed with Phenyl HP Sepharose™ hydrophobic interaction resin (Amersham Biosciences, Upsala, Sweden). The Q FTW (putatively comprising the antibodies of interest) was diluted with an equal volume of 1.7 M ammonium sulfate, 50 mM sodium phosphate, pH 7.0, then filtered through a 0.45/0.2 μm Sartopore™ bi-layer filter (1×30-inch). Phenyl Sepharose™ HP was run in two cycles with a maximum loading of 40 g ABT-874 per L of Phenyl Sepharose™ HP resin 3000 g per cycle).

Phenyl load was loaded on the column and equilibrated with five CVs of 0.85 M ammonium sulfate, 50 mM sodium phosphate, pH 7.0. Following the loading of product, the column was washed with three CVs of wash 1 (1.1 M ammonium sulfate, 50 mM sodium phosphate, pH 7.0), one to seven CVs of wash 2 (85 M ammonium sulfate, 50 mM sodium phosphate, pH 7.0), then three CVs of wash 3 (1.1 M ammonium sulfate, 50 mM sodium phosphate, pH 7.0).

The column was eluted with elution buffer, 0.5 M ammonium sulfate, 15 mM sodium phosphate, pH 7.0. The sample product was collected from the upside at 3 OD_{280 nm} to downside of peak at 3 OD_{280 nm}.

Equilibration, load, wash 1 wash 2, and wash 3 were performed at 6.2 L/min (linear velocity=74 cm/hr). Elution, regeneration, WFI wash, sanitize, and store column steps were performed at 3.1 L/min (linear velocity=37 cm/hr).

The column was regenerated between cycles with four CVs of Water For Injection (WFI), three CVs of 1 M NaOH, and four CVs of WFI between cycles. After the last cycle, the column was subjected to five CVs of storage buffer, 25 mM sodium phosphate, 20% IPA.

Two cycles of HIC were performed for each sample lot. The average load per cycle were 32.54 and 34.00 g/L resin (cycle A and cycle B, respectively); ranging from 21.77 to 37.79 g/L (see Tables 10 and 11). The overall product recovery yields for cycle A and B combined ranged from 81% to 86% with an average of 84% with a standard deviation of 1.87 for sample lots 29001BF, 29008BF, 30005BF, 30006BF, and 35036BF. A total of 20.94 Kg of ABT-874 was produced across all the aggregated Phenyl Sepharose HP chromatography steps.

Purity by SEC-HPLC for the Phenyl batches had an average of 99.63% for the IgG with a standard deviation of 0.11 (Table 16). The percent purity increased during the hydrophobic chromatography procedure with the purification of the antibodies of interest reducing the antibody related aggregate IgG and double light chain (low molecular weight species). This was achieved with a wash 2 buffer (SR-342: 25 mM sodium phosphate, 0.85 M ammonium sulfate, pH 7). This buffer elutes the low molecular weight species of double light chain antibody and is implemented after the A280 nm increases slightly and levels off at about one column volume with a flow rate that is half the load and wash 1 flow rate (flow rate reduction of 6.2 to 3.1 L per minute). Aggregates were removed by binding to Phenyl resin of the column and not eluting at the 0.5 M ammonium sulfate buffer criteria when the antibody was eluting.

TABLE 10
Phenyl Sepharose ™ HP chromatography data of sample lots
Phenyl Sepharose ™ HP chromatography

| | Purification sample lot # | | | | |
|---|---|---|---|---|---|
| Process step | 29001BF | 29008BF | 30005BF | 30006BF | 35036BF |
| *Q FTW volume (L) = undiluted | 369.2 | 441.9 | 380.2 | 507.90 | 365.5 |
| Phenyl load | | | | | |
| Q FTW concentration (g/L) | 13.81 | 12.74 | 13.92 | 10.95 | 8.97 |
| Total product loaded (g) | 5099 | 5630 | 5292 | 5562 | 3279 |
| 1.7 M Ammonium sulfate, 50 mM | 385 | 458.21 | 397.14 | 524.64 | 377.68 |
| sodium phosphate added to dilute Q | | | | | |
| FTW (Kg = L) | | | | | |
| Total volume Phenyl load (L) | 770 | 916.21 | 794.34 | 1049.34 | 755.08 |
| Cycle A | | | | | |
| Product loaded on column, cycle A | 2297 | 2834 | 2651 | 2786 | 1645 |
| (g) | | | | | |
| Volume load, cycle A (L) # from | 332.7 | 444.9 | 380.9 | 508.9 | 366.8 |
| chrom skid | | | | | |
| Actual loading of product/L of Phenyl | 30.6 | 37.79 | 35.21 | 37.15 | 21.93 |
| resin, cycle A (g product/L Phenyl | | | | | |
| resin) | | | | | |
| Volume Phenyl eluate, cycle A (L) | 136 | 136 | 139.8 | 130 | 134 |
| Cycle B | | | | | |
| Product loaded on column, cycle B | 2801 | 2796 | 2641 | 2775 | 1633 |
| (g) | | | | | |
| Volume load, cycle B (L) | 405.7 | 438.9 | 379.5 | 506.9 | 364.2 |
| Actual loading of product/L of Phenyl | 37.35 | 37.28 | 35.35 | 37.00 | 21.77 |
| resin, cycle B (g product/L Phenyl | | | | | |
| resin) | | | | | |
| Volume Phenyl eluate, cycle B (L) # | 139 | 138 | 141 | 129 | 134 |
| from chrom skid | | | | | |
| Cycle A + cycle B | | | | | |
| Load product cycle A + cycle B (g) | 5099 | 5630 | 5292 | 5562 | 3279 |
| Phenyl pooled eluate volume ( L) | 275 | 274 | 280.8 | 259 | 268 |
| Phenyl pooled eluate concentration | 15.58 | 17.19 | 16.02 | 18.56 | 9.86 |
| (g/L) | | | | | |
| Total product of pooled Phenyl | 4285 | 4710 | 4498 | 4807 | 2642 |
| eluate (g) | | | | | |
| Overall product yield for step (%) | 84 | 84 | 85 | 86 | 81 |

*The volume used is from the actual chromatography OIT volume reading during the cycle A and cycle B end of load for Phenyl Sepharose ™ HP.

TABLE 11
Analysis Phenyl Sepharose ™ HP chromatography data

| | Final Phenyl Sepharose ™ HP chromatography averages | | |
|---|---|---|---|
| | Five sample lots | | |
| Process step | AVG | SD | % CV |
| Q FTW volume (L) = undiluted Phenyl load | 413 | 61.42 | 14.87 |
| Q FTW concentration (g/L) | 12.10 | 2.11 | 17.4 |
| Total product (g) | 4972 | 970.4 | 19.52 |
| 1.7 M Ammonium sulfate, 50 mM sodium phosphate | 429 | 62.42 | 14.55 |
| added to dilute Q FTW (Kg = L) | | | |
| Total volume Phenyl load (L) | 857 | 124.88 | 14.57 |
| Cycle A | | | |
| Product loaded on column, cycle A (g) | 2441 | 491.9 | 20.15 |
| Volume load, cycle A (L) | 407 | 70.07 | 17.22 |
| Actual loading of product/L of Phenyl resin, cycle A | 32.54 | 6.563 | 20.17 |
| (g product/L Phenyl resin) | | | |
| Volume Phenyl eluate, cycle A (L) | 135 | 3.57 | 2.64 |
| Cycle B | | | |
| Product loaded on column, cycle B (g) | 2531 | 505.9 | 19.99 |
| Volume load, cycle B (L) | 419 | 56.71 | 13.53 |
| Actual loading of product/L of Phenyl resin, | 34.00 | 6.75 | 19.85 |
| cycle B (g product/L Phenyl resin) | | | |
| Volume Phenyl eluate, cycle B (L) | 136.2 | 4.764 | 3.5 |
| Cycle A + cycle B | | | |
| Load product cycle A + cycle B (g) | 4972 | 970.6 | 19.52 |
| Phenyl pooled eluate volume (L) | 271 | 8.27 | 3.05 |
| Phenyl pooled eluate concentration (g/L) | 15.40 | 3.33 | 21.6 |
| Total product of pooled Phenyl eluate (g) | 4189 | 887.4 | 21.18 |
| Overall product yield for step (%) | 84 | 1.87 | 2.23 |

Virus Filtration

The Phenyl eluate from the HIC step was filtered using an Ultipor DV50™ viral removal filtration step. The Ultipor DV50™ step provides for the physical removal of adventitious viruses ≧50 nm in diameter that may be present in the Phenyl Sepharose™ HP column eluate.

The hydrophobic interaction column eluate was passed through the pre-wet 0.1 μm filter and 2×30-inch Ultipor DV50™ filter train (Pall Corporation) at ≦34 psig. Post filtration, the filter was flushed with HIC elution buffer to remove any ABT-874 retained in the filter housing. The Ultipor DV50™ filtrate was stored in a pre-sterilized tank at 12° C.±2° C. prior to the final UF/DF formulation step.

Product recovery yield from the DV50™ filtration step ranged from 97% to 102% with an average yield of 100% for the various sample runs (see Tables 12 and 13). A total of 20.99 Kg of antibody product was processed for the five sample lots.

TABLE 12
Viral filtration data of sample lots
Virus filtration

| | Purification sample lot # | | | | |
|---|---|---|---|---|---|
| Process step | 29001BF | 29008BF | 30005BF | 30006BF | 35036BF |
| Phenyl pooled eluate volume Pre | 275 | 274 | 280.8 | 259 | 268 |
| DV50 ™ filtration (Kg = L) | | | | | |
| Phenyl pooled eluate concentration | 15.58 | 17.19 | 16.02 | 18.56 | 9.86 |
| (g/L) | | | | | |
| Total product of pooled Phenyl | 4285 | 4710 | 4498 | 4807 | 2642 |
| eluate (g) | | | | | |
| 0.5 M Ammonium sulfate, 50 mM | 45.3 | 41.7 | 43.7 | 46.5 | 43.3 |
| sodium phosphate added to flush | | | | | |
| filters (Kg) | | | | | |
| Total viral filtrate + buffer flush (Kg) | 337.2 | 324.2 | 336.2 | 313.4 | 324.4 |
| Total viral filtrate (L)* | 324.23 | 311.73 | 323.27 | 301.35 | 311.92 |
| Viral filtrate concentration (g/L) | 13.27 | 15.20 | 14.22 | 15.45 | 8.66 |
| Total product of viral filtrate (g) | 4303 | 4738 | 4597 | 4656 | 2701 |
| Overall product yield for step (%) | 100 | 101 | 102 | 97 | 102 |

*Correction for density of 0.5 M ammonium sulfate, 50 mM sodium phosphate —divide Kg weight by density 1.04 g/mL = volume in L.

TABLE 13
Analysis virus filtration data

| | Final virus filtration averages | | |
|---|---|---|---|
| | Five sample lots | | |
| Process step | AVG | SD | % CV |
| Total viral filtrate (L) | 271 | 8.27 | 3.05 |
| Viral filtrate concentration (g/L) | 15.40 | 3.33 | 21.6 |
| Total product of viral filtrate (g) | 4189 | 887.4 | 21.18 |
| 0.5 M Ammonium sulfate, 50 mM sodium | 44.1 | 1.85 | 4.2 |
| phosphate added to flush filters (Kg) | | | |
| Total viral filtrate + buffer flush (Kg) | 327.1 | 9.85 | 3.01 |
| Total viral filtrate (L) | 314.5 | 9.47 | 3.01 |
| Viral filtrate concentration (g/L) | 13.40 | 2.77 | 20.7 |
| Total product of viral filtrate (g) | 4199 | 853.2 | 20.32 |
| Overall product yield for step (%) | 100 | 2.07 | 2.07 |

Final Ultrafiltration/Diafiltration (UF/DF):

The final UF/DF step was the concentration of antibody, removal of ammonium sulfate and the formulation of antibody product in 5 mM histidine, 5 mM methionine, 2% mannitol, 0.5% sucrose, 0.005% Tween 80, pH 5.9. Millipore Corporation regenerated cellulose ultrafiltration type membrane cassettes with a 30 kD molecular weight cut-off (MWCO) were used for this step.

The Ultipor DV50™ filtrate was concentrated to 30 g/L protein. Continuous diafiltration with two volumes of 5 mM methionine, 2% mannitol, 0.5% sucrose, pH 5.9 buffer (no Tween) was performed. The product was then concentrated to 40 g/L now that most of the ammonium sulfate was removed. Continuous diafiltration with six volumes of 5 mM methionine, 2% mannitol, 0.5% sucrose, pH 5.9 buffer (no Tween) was performed. Upon completion of these six diavolume exchange, the antibody was concentrated to 75 g/L. The UF system was then drained of product and rinsed with diafiltration buffer to recover product held up in the system. The concentrate and wash were combined to produce the diafiltered ABT-874 and was subsequently adjusted to ≧65 g/L with additional formulation buffer. Once the target concentration had been confirmed, a calculation was performed to determine the quantity of formulation buffer containing 10% Tween 80 that must be added to the concentrated UF retentate to bring the final Tween 80 concentration in drug substance to 0.005% (v/v). Final concentration of formulated drug substance was ≧65 g/L. The antibody sample was filtered through an OpticapXLT30™ capsule 2.0/1.2 μm filter followed with a 0.45/0.2 μm Sartopore™ bi-layer filter into a sterile container, then transferred to a Class 100 area in preparation for final bottling.

Product recovery yield from the UF/DF step ranged from 94% to 100% with an average yield of 97.0% with a standard deviation of 2.22 for five runs. (See Tables 14 and 15.) There was a total of 20.51 Kg of antibody product at the end of this Final UF/DF. Overall, the UF/DF filtration step was very consistent run to run. Use of the 30 kD cutoff membrane rather than a 10 kD cutoff membrane allowed for faster processing time without sacrificing yield.

TABLE 14
Final UF/DF data of sample lots
Final UF/DF

| | Purification sample lot # | | | | |
|---|---|---|---|---|---|
| Process step | 29001BF | 29008BF | 30005BF | 30006BF | 35036BF |
| Viral filtrate (L) | 324.23 | 311.73 | 323.27 | 301.35 | 311.92 |
| Viral filtrate concentration (g/L) | 13.27 | 15.20 | 14.22 | 15.45 | 8.66 |
| Total product of viral filtrate (g) | 4303 | 4738 | 4597 | 4656 | 2701 |
| First diafiltration working volume | 135 | 150 | 145 | 147 | 82 |
| TK-2575 (L) | | | | | |
| First diafiltration buffer volume (L) | 291 | 318 | 315 | 311 | 216 |
| Second diafiltration working volume | 107.3 | 118.4 | 107.1 | 120 | 60.1 |
| TK-2575 (L) | | | | | |
| Second diafiltration buffer volume (L) | 694 | 730 | 663 | 770 | 450 |
| Measured pH pre-transfer from | 5.8 | 5.8 | 5.9 | 5.8 | 6.0 |
| UF system | | | | | |
| Measured conductivity pre-transfer | 0 | 3 | 0 | 0 | 0 |
| from UF system (mS/cm) | | | | | |
| Post transfer UF/DF filtrate (Kg) | 67.3 | 71.2 | 71.2 | 68 | 44.8 |
| 1% Tween, formulation buffer added | 0.337 | 0.356 | 0.356 | 0.340 | 0.822 |
| (L) | | | | | |
| Filtered final UF/DF retentate in L | 65.1 | 71.2 | 68.4 | 66 | 42.4 |
| (Kg) | | | | | |
| Final UF/DF retentate concentration | 65.70 | 66.48 | 63.90 | 69.70 | 59.66 |
| (g/L) | | | | | |
| Total product of Final UF/DF | 4277 | 4733 | 4371 | 4600 | 2530 |
| retentate (g) | | | | | |
| Overall product yield for step (%) | 99 | 100 | 95 | 99 | 94 |

TABLE 15
Analysis final UF/DF data

| | Final UF/DF averages | | |
|---|---|---|---|
| | Five sample lots | | |
| Process step | AVG | SD | % CV |
| Viral filtrate volume (Kg = L) | 314.5 | 9.47 | 3.01 |
| Viral filtrate concentration (g/L) | 13.4 | 2.77 | 20.7 |
| Total product of viral filtrate (g) | 4199 | 853.2 | 20.32 |
| First diafiltration working volume | 131.8 | 28.4 | 21.55 |
| TK-2575(L) | | | |
| First diafiltration buffer volume (L) | 290.2 | 42.8 | 14.75 |
| Second diafiltration working volume | 102.6 | 24.5 | 23.88 |
| TK-2575 (L) | | | |
| Second diafiltration buffer volume (L) | 661.4 | 124.75 | 18.86 |
| Measured pH pre-transfer from UF system | 5.86 | 0.089 | 1.52 |
| Measured conductivity pre-transfer from UF | 0.6 | 1.342 | 223.67 |
| system (mS/cm) | | | |
| Post transfer UF/DF filtrate (Kg) | 64.5 | 11.16 | 17.3 |
| 1% Tween, formulation buffer added (L) | 0.44 | 0.212 | 48.18 |
| Filtered final UF/DF retentate in L (Kg) | 62.6 | 11.55 | 18.45 |
| Final UF/DF retentate concentration (g/L) | 65.1 | 3.69 | 5.7 |
| Total product of final UF/DF retentate (g) | 4102 | 208.8 | 5.09 |
| Overall product yield for step (%) | 97 | 2.22 | 2.29 |

TABLE 16
Analysis of five sample lots in-process QC analytical samples

| | Test | Method | Specification | AVG | SD | % CV |
|---|---|---|---|---|---|---|
| Cell | | | | | | |
| culture/process | | | | | | |
| steps | | | | | | |
| Production | Poros A | QCA-228 | Report value g/L | 3.91 | 0.12 | 3.07 |
| reactor harvest | HPLC | | | | | |
| sample | | | | | | |
| Post depth pH | Poros A | QCA-228 | Report value g/L | 3.46 | 0.13 | 3.76 |
| filtration | HPLC | | | | | |
| inactivation | | | | | | |
| Clarified harvest | Poros A | QCA-228 | Report value g/L | 3.18 | 0.1 | 3.14 |
| | HPLC | | | | | |
| Purification/process | | | | | | |
| steps | | | | | | |
| CM HyperDF | Poros A | QCA-228 | Poros A HPLC | 30.9 | 5.18 | 16.76 |
| eluate | HPLC | | g/L | | | |
| | SE-HPLC | QCA-232 | Percent purity | 94.76 | 0.67 | 0.71 |
| | | | Report value | | | |
| Concentrated CM | A280 | QCA-227-01 | Report value g/L | 37.13 | 3.61 | 9.72 |
| HyperDF ™ eluate | SE-HPLC | QCA-232 | Percent purity | 95.79 | 0.57 | 0.6 |
| | | | Report value | | | |
| Q Sepharose ™ | A280 | QCA-227-01 | Report value g/L | 12.08 | 2.11 | 17.47 |
| flow through | SE-HPLC | QCA-232 | Percent purity | 96.75 | 0.53 | 0.55 |
| wash | | | Report value | | | |
| Phenyl | A280 | QCA-227-01 | Report value g/L | 15.44 | 3.33 | 21.57 |
| Sepharose ™ HP | SE-HPLC | QCA-232 | Percent purity | 99.63 | 0.11 | 0.11 |
| eluate | | | Report value | | | |
| Ultipor ™ VF | A280 | QCA-227-01 | Report value g/L | 13.36 | 2.77 | 20.73 |
| filtrate | SE-HPLC | QCA-232 | Percent purity | 99.63 | 0.09 | 0.09 |
| | | | Report value | | | |
| Formulated final | A280 | QCA-227-01 | Report value g/L | 65.09 | 3.69 | 5.67 |
| UF/DF retentate | SE-HPLC | QCA-232 | Percent purity | 99.45 | 0.13 | 0.13 |

### 2. Determination of Host Cell Protein Concentration in Anti-IL-12 Antibody Compositions

This procedure describes the testing methodology for the determination of residual Host Cell Protein concentration in anti-IL-12 antibody samples. Enzyme Linked Immunosorbent Assay (ELISA) is used to sandwich the Host Cell Protein (Antigens) between two layers of specific antibodies. This is followed by the blocking of non-specific sites with Casein. The Host Cell Proteins are then incubated during which time the antigen molecules are captured by the first antibody (Coating Antibody). A second antibody (anti- Host Cell Protein Biotinylated) is then added which fixes to the antigen (Host Cell Proteins). Neutravidin HRP-conjugated is added which binds to the Biotinylated anti-Host Cell Protein. This is followed by the addition of K blue substrate. The chromogenic substrate is hydrolyzed by the bound enzyme conjugated antibody, producing a blue color. Reaction is stopped with 2M H₃PO₄, changing color to yellow. Color intensity is directly proportional to the amount of antigen bound in the well.

Preparation of 50 mM Sodium Bicarbonate (Coating Buffer), pH 9.4. To a 1 L beaker add: 900 mL Milli-Q water; 4.20 g±0.01 g Sodium Bicarbonate. Stir until completely dissolved. Adjust pH to 9.4 with 1 N NaOH. Transfer to a 1 L volumetric flask and bring to volume with Milli-Q water. Mix by inversion until homogeneous. Filter through a 0.22 μm sterile filter unit. Store at nominal 4° C. for up to 7 days from the date of preparation.

Preparation of 0.104 M Na₂HPO₄*7H₂O, 1.37 M NaCl, 0.027 M KCl, 0.0176 M KH₂PO₄, pH=6.8-6.9 (10× PBS). Add approximately 400 mL of Milli-Q water to a glass beaker. Add 13.94 g±0.01 g of Na₂HPO₄×7H₂O. Add 40.0 g±0.1 g of NaCl. A 1.00 g±0.01 g of KCl. Add 1.20 g±0.01 g of KH₂PO₄. Stir until homogeneous. Transfer to a 500 mL volumetric flask. QS to 500 mL volume with Milli-Q water. Mix by inversion. Filter through a 0.2 μm sterile filter unit. Store at room temperature for up to 7 days.

Preparation of 1× PBS+0.1% Triton X-100, pH 7.40: (Plate Wash Buffer). In a 4 L graduated cylinder, mix 400 mL 10 X PBS (step 5.2) with 3500 mL Milli-Q Water. Check pH, and adjust if necessary to 7.40±0.05 with 1 N HCl or 1 N NaOH. Bring to volume with Milli-Q water. Tightly parafilm the cylinder and mix by inversion until homogeneous. Transfer to a 4 L bottle. Remove 4 mL of the 1×PBS and discard. Add 4 mL of triton X-100 to the 3996 mL of 1×PBS. Place on stir plate and stir to completely dissolve. Filter the amount of plate wash buffer needed for dilution buffer preparation through a 0.22 μm sterile filter unit. Store at room temperature for up to 7 days.

Preparation of Coating Antibody Mixture: goat anti CHO 599/626/748 (lot #G11201@1.534 mg/mL), affinity purified: NOTE: Stocks stored at nominal −80° C. in vials. Prepare aliquots. Take out one aliquot per plate at time of use. Immediately before use: Dilute antibody mixture to have a final concentration of 4 μg/mL in cold 50 mM Sodium Bicarbonate as follows. For example: add 31 μLs coating antibody mixture to 11969 μLs cold coating buffer. Mix gently by inversion.

Preparation of Biotinylated goat anti Host Cell Protein Mixture, 599/626/748 (lot#G11202@0.822 mg/mL): NOTE: Stocks stored at nominal −80° C. in vials. Prepare aliquots. Take out one aliquot per plate at time of use. Immediately before use: dilute biotinylated antibody mixture to have a final concentration of 1 μg/mL in 37° C.±2° C. Casein as follows. For example: add 14.6 μLs biotinylated antibody mixture to 11985 μLs 37° C.±2° C. Casein. Mix gently by inversion.

Preparation of Neutravidin-HRP. Reconstitute new lots (2 mg/vial) to 1 mg/mL as follows: Add 400 μL of Milli-Q water to the vial, then add 1600 μL 1× PBS, for a total of 2 mL. Vortex gently to mix. Store at nominal −20° C. Prepare aliquots with desired volume so that 1 aliqout per plate is used. Prepare in polypropylene tube. Qualify new lots to determine working concentration. Assign expiry of 6 months from the date of preparation. For example, if the working concentration was determined to be 0.2 μg/mL then prepare as follows. Immediately before use: thaw an aliquot of Neutravidin-HRP at room temperature. Dilute the 1 mg/mL Neutravidin solution to 0.1 mg/mL (100 μg/mL) with 37° C.±2° C. Casein. For example: Dilute X10, add 50 μL of neutravidin to 450 μL of Casein. Vortex gently to mix. Further dilute the 100 μg/mL solution to 0.2 μg/mL with 37° C.±2° C. Casein. For example: Dilute X500, add 24 μL neutravidin (100 μg/mL) to 11976 μL of Casein. Vortex gently to mix.

Preparation of 5.7 2M Phosphoric Acid (Stop Solution). Prepare a 2 M Phosphoric acid solution from concentrated phosphoric acid as follows. From the % phosphoric acid stated on the label, density (1.685 g/mL) and formula weight (98 g/mole), calculate the volume of concentrated phosphoric acid needed to prepare 500 mL of 2M phosphoric acid. Add the volume of concentrated phosphoric acid calculated above to the flask. Bring to volume with Milli-Q water and mix by inversion until homogeneous. Store at ambient temperature for up to 6 months from the date of preparation.

Preparation of Dilution Buffer (Casein diluted X100 in 1× PBS +0.1% Triton X100, pH 7.4). Dilute 37° C.±2° C. Casein X100 in 0.22 μm sterile filtered 1× PBS+0.1% Triton X100, pH 7.4 (from above). For example: Add 1 mL of 37° C.±2° C. Casein to 99 mL 0.22 μm sterile filtered 1× PBS+0.1% Triton X100, pH 7.4. Mix well. Prepare fresh for each use.

Preparation of Standards. Host cell Protein Standards (Antigen Standards) (lot #G11203@1.218 mg/mL): NOTE: Stocks stored at nominal −80° C. in 70 μL aliquots. Thaw an aliquot at room temperature. Perform serial dilutions in polypropylene tubes using Dilution buffer.

Preparation of Samples. In polypropylene tubes, dilute final bulk samples to 24 mg/mL in Dilution Buffer. Record concentration. NOTE: use the solutions below to prepare spiked samples and to prepare the 12 mg/mL solutions referenced below. In polypropylene microtubes, further dilute the 24 mg/mL solutions to 12 mg/mL in Dilution Buffer. Load triplicate wells for each of the 12 mg/mL solutions on the plate for a total of 6 wells.

Preparation of Spike. In a polypropylene microtube, prepare a 10 ng/mL Host Cell Protein spike from the 20 ng/mL standard prepared above by diluting it 2× with Dilution Buffer. Load three wells for the 10 ng/mL spike solution onto the plate. Use the 20 ng/mL standard solution from step 6.1 for spiking samples.

Preparation of Spiked Samples. In polypropylene microtubes, spike 300 μL of each 24 mg/mL final bulk solution with 300 μL of the 20 ng/mL spike solution (6.1). Load triplicate wells for each spiked sample solution for a total of 6 wells.

Preparation of Control. A control range must be set for every new control stock solution, before use in routine testing. Control Stock: Prepare 150 μL aliquots of a batch of ABT-874 Drug Substance Concentrate and store frozen at nominal −80° C. for up to three years.

Preparation of Working Control. Thaw an aliquot of control at room temperature. In polypropylene tubes, dilute control to 24 mg/mL with Dilution Buffer. In polypropylene microtubes, further dilute the 24 mg/mL control solution with dilution buffer to 12 mg/mL. Prepare a single dilution and load control into 3 wells of the plate.

ELISA procedures. Fill plate wash bottle with plate wash buffer (refer to step 5.3, 1× PBS+0.1% Triton X-100). Prime plate washer. Check the following parameters: Parameters should be set to: Plate Type: 1 For each Cycle (a total of 5 cycles): Volume: 400 μls; Soak Time: 10 seconds; Asp. Time: 4 seconds.

Assay Procedure. Coat plates with 100 μL/well of 4 μg/mL goat coating antibody mixture in cold 50 mM Sodium Bicarbonate. Tap the side of the plate until the coating solution covers the bottom of the wells uniformly, cover with sealing tape and incubate at nominal 4° C. while shaking on plate shaker (or equivalent) at speed 3 for 18 hours±1 hour. After overnight incubation, remove plate from refrigerator and allow to equilibrate to room temperature. Shake out coating. Blot plate on paper towels. Block with 300 μL/well of 37° C.±2° C. Casein, cover with sealing tape and incubate at 37° C.±2° C. while shaking on Lab-line Environ plate shaker (or equivalent) at 80 rpm±5 rpm for 1 hour. Prepare standard, sample, control, spike, and spiked samples during blocking incubation. Wash the plate 5 times with Wash Buffer. Blot plate on paper towels. Using an 8-channel pipette, pipet 100 μL/well of standards, samples, spikes, spiked samples, and control into triplicate wells of the plate. Pipette 100 μL/well of Dilution Buffer into all empty wells of the plate to serve as blanks. Cover with sealing tape and incubate at 37° C.±2° C. while shaking on Lab-line Environ plate shaker (or equivalent) at 80 rpm±5 rpm for 1 hour. Fill out a template to use as a guide when loading plate.

Plate Reader Set-Up. Set up template, entering concentrations for standards. Do not enter dilution factors for samples, control, spike, or spiked samples. Assign the wells containing diluent as blanks to be subtracted from all wells. Wash the plate 5 times with Wash Buffer. Blot plate on paper towels. Add 100 μL/well biotinylated goat antibody. Cover with sealing tape and incubate at 37° C.±2° C. while shaking on Lab-line Environ plate shaker (or equivalent) at 80 rpm±5 rpm for 1 hour. Wash the plate 5 times with Wash Buffer. Blot plate on paper towels. Add 100 μL/well Neutravidin-HRP conjugate solution. Cover with sealing tape and incubate at 37° C.±2° C. while shaking on Lab-line Environ plate shaker (or equivalent) at 80 rpm±5 rpm for 1 hour. Wash the plate 5 times with Wash Buffer. Blot plate on paper towels. Add 100 μL/well cold K-Blue substrate, cover with sealing tape and incubate at room temperature for 10 minutes (start timer as soon as substrate is added to first row), while shaking speed 3 on Lab-line titer plate shaker (or equivalent). Stop the reaction by adding 100 μL/well 2M Phosphoric Acid (Step 5.7). Place plate on a plate shaker at speed 3 for 3-5 minutes. Read plate at 450 nm.

Data Analysis and Calculations. NOTE: only samples, spikes, spiked samples, and control, with optical densities falling within the practical quantitation limit (2.5 ng/mL standard) of the standard curve and meeting the % CV or % difference criteria stated below, are accepted. If sample OD's fall below the 2.5 ng/mL standard, result should be reported as less than 2.5 ng/mL. This value should then be divided by the diluted sample concentration (12 mg/mL) to report value in ng/mg. If sample is high in host cell concentration causing the non-spiked and/or the spiked sample to be above standard curve, report value as >100 ng/mL. This value should then be divided by the diluted sample concentration (12 mg/mL) to report value in ng/mg. Consider sample value zero for spike recovery calculations when the sample is below the 2.5 ng/mL standard.

102001 Standard Curve. Standard concentrations should be entered into the protocol template. A quadratic curve fit is used. Coefficient of determination must be=0.99 and the % CV between triplicate wells must be=20%. If this criteria is not met: One standard (1 level, 3 wells) may be dropped. If the 1.25 ng/mL is dropped, only samples and spiked samples with optical densities falling within the 2.5 ng/mL and 100 ng/mL (the remaining standard curve points) optical densities are acceptable. Additionally, for the triplicates of each standard level, if a single well is clearly contaminated or shows low binding, it may be dropped. If a well is dropped from a standard level, the remaining replicates must have a % difference=20%. The % CV for the lowest standard, which shows OD values close to the background (blanks) of the plate, should be=30%. If one well is dropped, the % difference for the remaining replicates must be=35%. If the lowest standard is dropped, only samples and spiked samples with optical densities falling within the remaining standard curve level optical densities are acceptable.

Samples. % CV should be=20% between triplicate wells. Report % CV between triplicate wells. One well from each sample dilution may be dropped. The remaining replicates must have a % difference of=20%. Note: if non-spiked sample OD is below the 2.5 ng/mL standard OD the % difference criteria does not apply to the non-spiked results. Refer to calculation above.

Calculate actual Host Cell Concentration in ng/mg from the mean (ng/mL) value as follows: CHO Host Cell Protein (ng/mg)=Mean “Non-spiked sample result (ng/mL)”_Diluted sample concentration (12 mg/mL).

Spikes. % CV should be=20% between triplicate wells. Record % CV. One well from the spike may be dropped. The remaining points must have a % difference=20%. Refer to calculation in above. Report host cell concentration in ng/mL. This result will be used in spike recovery calculations. The resulting concentration for the spike (ng/mL) must be ±20% of the theoretical spike concentration. Record result and indicate Pass or Fail. If the spike result is not within 20% of theoretical, the assay must be repeated. Mean Spike Concentration (ng/mL)×100=must be 100%±20% 10 ng/mL.

Spiked Samples. % CV should be=20% between triplicate wells. Record % CV between triplicate wells. One well from each spiked sample dilution may be dropped. The remaining replicates must have a % difference of=20%. Refer to calculation above. Report “Spiked sample result” for each dilution in ng/mL. Record % difference between duplicate dilutions. The % difference between dilutions should be=25%. These results will be used in the spike recovery calculations.

Calculate % Spike Recovery for each dilution set using the formula below: % Spike Recovery=Spiked sample value−Non-Spiked Sample Value X 100 Spike Value. NOTE: (1) If non-spiked sample value OD's fall below the 2.5 ng/mL standard consider value as zero in % spike recovery calculation. % Spike recovery must be 100%±50% (50% −150%) for each dilution for each sample. Record results and Pass/Fail.

Control. % CV should be=20% between triplicate wells. Record % CV result. One well from the control may be dropped. The remaining replicates must have a % difference of=20%. Refer to calculation above. Report Host Cell concentration in the control in ng/mL. Calculate Host Cell concentration in ng/mg as follows: Host Cell Protein (ng/mg)=Control Host Cell Protein result in ng/mL.

Various publications are cited herein, the contents of which are hereby incorporated by reference in their entireties.

What is claimed is:

1. A method for producing a host cell-protein (HCP) reduced antibody preparation from a sample mixture comprising an antibody and at least one HCP, said method comprising:
(a) subjecting said sample matrix to a reduction in pH thus forming a primary recovery sample, wherein said reduction in pH is to between 3 and 4;
(b) adjusting said primary recovery sample to a pH of between about 4.5 and 6 followed by applying said primary recovery sample to an ion exchange resin and collecting an ion exchange sample;
(c) applying said ion exchange sample to a hydrophobic interactive chromatography (HIC) resin and collecting an HIC sample, wherein said HIC sample comprises said HCP-reduced antibody preparation.

2. The method of claim 1, wherein said reduction in pH is accomplished by admixing a suitable acid with said sample mixture, and wherein said suitable acid is selected from the group consisting of citric acid, acetic acid, caprylic acid, and the like.

3. The method of claim 1, wherein said ion exchange resin is either an anion exchange resin or a cation exchange resin.

4. The method of claim 3, wherein said ion exchange resin is a cation exchange resin.

5. The method of claim 4, wherein said cation exchange resin is selected from the group consisting of carboxymethyl (CM), sulfoethyl(SE), sulfopropyl(SP), phosphate(P) and sulfonate(S).

6. The method of claim 5, wherein said cation exchange resin is carboxymethyl.

7. The method of claim 3, wherein said ion exchange resin is an anion exchange resin.

8. The method of claim 7, wherein said anion exchange resin is selected from the group consisting of Q sepharose, diethylaminoethyl (DEAE), quaternary aminoethyl(QAE), and quaternary amine(Q) groups.

9. The method of claim 8, wherein said anion exchange resin is Q-sepharose.

10. The method of claim 1, wherein said ion exchange step comprises a first ion exchange step and a second ion exchange step.

11. The method of claim 10, wherein said first ion exchange step is a cation exchange step followed by a second anion exchange step.

12. The method of claim 10 further comprising an intermediate step, wherein said intermediate step is a filtration step occurring between said first and said second ion exchange step.

13. The method of claim 12, wherein said filtration step is accomplished by capture ultrafiltration/diafiltration.

14. The method of claim 1, wherein said HIC is accomplished using a column comprising one or more hydrophobic groups.

15. The method of claim 14, wherein said one or more hydrophobic groups are selected from the group consisting of alkyl-, aryl-groups, and a combination thereof.

16. The method of claim 14, wherein said column is selected from the group consisting of phenyl sepharose (such as Phenyl Sepharose™ 6 Fast Flow column, Phenyl Sepharose™ High Performance column), Octyl Sepharose™ High Performance column, Fractogel™ EMD Propyl, Fractogel™ EMD Phenyl columns, Macro-Prep™ Methyl, Macro-Prep™ t-Butyl Supports, WP HI-Propyl (C₃)™ column, and Toyopearl™ ether, phenyl or butyl columns.

17. The method of claim 16, wherein said column comprises phenyl sepharose.

18. The method of claim 1 further comprising a filtration step, wherein said HIC sample is subjected to filtration to remove viral particles and to facilitate buffer exchange.

19. The method of claim 1, wherein said HCP-reduced antibody preparation comprises an anti-IL-12 antibody or an antigen-binding portion thereof.

20. The method of claim 19, wherein said anti-IL-12 antibody or antigen-binding portion thereof is a humanized antibody, a chimeric antibody, or a multivalent antibody.

21. The method of claim 20, wherein said anti-IL-12 antibody or antigen-binding portion thereof is a humanized antibody.

22. The method of claim 20, wherein said anti-IL-12 antibody or antigen-binding portion thereof is an isolated human antibody that dissociates from human IL-12 with a K_{d} of about 1×10⁻⁸ M or less and a K_{off} rate constant of about 1×10⁻³ s⁻¹ or less both determined by surface Plasmon resonance.

23. The method of claim 19, wherein said anti-IL-12 antibody or antigen-binding portion thereof neutralizes IL-12 both in vivo and in vitro.

24. The method of claim 1, wherein said preparation is substantially free of HCPs.

25. A method for producing a host cell-protein (HCP) reduced antibody preparation from a sample mixture comprising an antibody and at least one HCP, said method comprising:
(a) subjecting said sample matrix to a reduction in pH thus forming a primary recovery sample, wherein said reduction in pH is to about 3.5;
(b) adjusting said primary recovery sample to a pH of about 4.9 followed by applying said primary recovery sample to a cation exchange resin and collecting a cation exchange sample;
(c) applying said cation exchange sample to an anion exchange resin and collecting a anion exchange sample; and
(d) applying said anion exchange sample to a hydrophobic interactive chromatography (HIC) resin and collecting an HIC sample, wherein said HIC sample comprises said HCP-reduced antibody preparation.

26. A method for producing a host cell-protein (HCP) reduced antibody preparation from a sample mixture comprising an antibody and at least one HCP, said method comprising:
(a) subjecting said sample matrix to a reduction in pH thus forming a primary recovery sample, wherein said reduction in pH is to about 3.5;
(b) adjusting said primary recovery sample to a pH of about 4.9 followed by applying said primary recovery sample to a cation exchange resin and collecting a cation exchange sample;
(c) subjecting said cation exchange sample to filtration and collecting a filtrate.
(d) applying said filtrate from (c) to an anion exchange resin and collecting an anion exchange sample; and
(e) applying said anion exchange sample to a hydrophobic interactive chromatography (HIC) resin and collecting an HIC sample, wherein said HIC sample comprises said HCP-reduced antibody preparation.

27. A pharmaceutical composition comprising an HCP-reduced antibody preparation produced by the method of claim 1 and a pharmaceutically acceptable carrier.

28. The pharmaceutical composition of claim 27, wherein said antibody is an anti-IL-12 antibody or antigen-binding portion thereof.

29. The pharmaceutical composition of claim 27, wherein said composition is substantially free of HCPs.

30. The pharmaceutical composition of claim 27 used to neutralize IL-12 facilitated disorders.

31. The pharmaceutical composition of claim 30, wherein said disorders are selected from the group consisting of rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anemia, Coombs positive haemolytic anaemia, acquired pernicious anemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, and autoimmune uveitis.

32. The pharmaceutical composition of claim 27 further comprising a non-steroidal or steroidal anti-inflammatory drug.

33. The pharmaceutical composition of claim 32 comprising a non-steroidal anti-inflammatory drug.

34. The pharmaceutical composition of claim 33, wherein said non-steroidal anti-inflammatory drug is selected from the group consisting of ibuprofen, corticosteroids, prednisolone,

35. The pharmaceutical composition of claim 32 comprising a steroidal anti-inflammatory drug.

36. The pharmaceutical composition of claim 27 further comprising one or more other antibodies or antigen-binding portions thereof.

37. The pharmaceutical composition of claim 27 further comprising a pharmaceutical agent.

38. The pharmaceutical composition of claim 37, wherein said pharmaceutical agent is selected from the group consisting of methotrexate, 6-MP, azathioprine sulphasalazine, mesalazine, olsalazine chloroquinine/hydroxychloroquine, pencillamine, aurothiomalate, azathioprine, cochicine, corticosteroids, β-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, phosphodiesterase inhibitors, adensosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™)and p55TNFRIgG (Lenercept), sIL-1 RI, sIL-1RII, sIL-6R, soluble IL-13 receptor (sIL-13)) and anti-inflammatory cytokines (e.g., IL-4, IL-10, IL-11, IL-13 and TGFβ).

39. The methods of claims 1, 25 and 26, wherein said HCP-reduced antibody preparation comprises one or more anti-IL-12 antibodies or antigen-binding portions thereof and are labeled.

40. The methods of claim 39, wherein said label is radioactive.

41. The methods of claim 40, wherein said radioactive label is selected from the group consisting of ¹²⁵I, ¹³¹I, ³⁵S, and ³H.

42. The methods of claim 39, wherein said label is non-radioactive.

43. The methods of claims 1, 25, and 26, wherein said HCP-reduced antibody preparation comprises one or more anti-IL-12 antibodies or antigen-binding portions thereof and are pegylated.


SEQUENCE LISTING (2 sequences)

SEQ ID NO: 1 - protein, 115 aa, Artificial sequence
  Synthetic polypeptide
QVQLVESGGGVVQPGRSLRLSCAASGFTFSSYGMHWVRQAPGKGLEQVAFIRYDGSNKYYADSVKGRFTISRDNSKNTLYLQMNSLRAEDTAVYYCKTHGSHDNWGQGTMVTVSS

SEQ ID NO: 2 - protein, 112 aa, Artificial sequence
  Synthetic polypeptide
QSVLTQPPSVSGAPGQRVTISCSGSRSNIGSNTVKWYQQLPGTAPKLLIYYGNDQRPSGVPDRFSGSKGTSASLAITGVQAEDEADYYCQSYDRYTHPALLFGTGTKVTVLG